(19) United States
(12) Patent Application Publication
(10) Pub. No.: US 2023/0230698 A1
(43) Pub. Date: Jul. 20, 2023

(54) ANTIMICROBIAL CHOICE ALGORITHM FOR URINARY TRACT INFECTIONS

(71) Applicant: Northwestern University, Evanston, IL (US)

(72) Inventors: Anthony J. Schaeffer, Hinsdale, IL (US); Jason E. Cohen, Chicago, IL (US)

(21) Appl. No.: 18/007,111

(22) PCT Filed: Jul. 26, 2021
(86) PCT No.: PCT/US2021/070959

§ 371 Date: Jan. 27, 2023

Related U.S. Application Data
(60) Provisional application No. 63/057,031, filed on Jul. 27, 2020.

(51) Int. Cl. G16H 50/20; G16H 50/70; G16H 20/10; G16H 70/40; G16H 10/60

(52) U.S. Cl. CPC G16H 50/20; G16H 50/70; G16H 20/10; G16H 70/40; G16H 10/60

(57) ABSTRACT

Disclosed herein are systems and methods for significantly improving the accuracy of drug selection for patients diagnosed with, or symptomatic for, urinary tract infections (UTIs). In some embodiments, the systems and methods utilize real-time patient data linked to geographic distribution maps of resistance to different antimicrobial treatments for UTIs. The combination of real-time date and resistance maps allow for more accurate selection of appropriate UTI therapy for the patient.

### CROSS-REFERENCE TO RELATED APPLICATIONS

The present application claims the benefit of U.S. Application No. 63/057,031 filed on Jul. 27, 2020, the contents of which are incorporated herein by reference in their entirety.

### BACKGROUND

Symptomatic urinary tract infections (UTIs) are among the most common bacterial infections accounting for more than 7 million outpatient visits to physicians' offices and well over one million hospital admissions in the United States annually. In ambulatory patients alone, the national health care cost of uncomplicated lower UTIs is estimated to approach $1 billion. At a time when controlling spiraling health care costs is a national priority, improvements in the management of UTI is imperative.

Currently, when a patient pays an office visit to the doctor complaining of the symptoms of UTI, a presumptive diagnosis is made by examining the urine microscopically for microorganisms or by performing an indirect dipstick test that measures a microbial metabolite. Although these tests may reveal the presence of a microbial infection, they do not identify the type of bacterium present or indicate the drug sensitivity of the bacterium. Moreover, a urine culture is not typically performed if the patient appears to have an uncomplicated UTI. Rather, a short course of empirical antimicrobial therapy is given. No follow-up visit or bacterial culture after therapy is necessary unless symptoms persist or recur. If any clinical features or other factors suggest a complicated infection, a bacterial culture is indicated and should be performed before therapy is started. Mitigating factors that would necessitate a culture include diabetes, symptoms for greater than seven days, recent UTI or antimicrobial use, use of a diaphragm, age less than thirteen or greater than sixty-five, and pregnancy.

In principle, the most appropriate antimicrobial to treat an infection could be determined by a clinical microbiology laboratory. That is, the infectious microorganism may be cultured, identified, and tested for drug sensitivity. In practice, this is rarely done because of the expense and because the demand for relief from the symptoms of UTI is too urgent to wait for results of such prolonged testing. As a result of the urgency for prompt treatment, antibiotics are generally prescribed empirically and with variable success.

This empirical method of treating patients without cultures or documentation has significant potential for complications and needless treatment of patients who do not have infections. Approximately ninety-five percent of patients with UTI have no serious risk of complications and empirical treatment is sufficient. However, about five percent have significant risk for complications which can be costly to treat and which can even be life threatening. For instance, if a patient is treated with empirically prescribed antimicrobials for a presumptive infection when in fact she has another more serious condition such as bladder cancer (which often has the same symptoms as UTI), obvious adverse results can ensue from a delay in diagnosis. However, the alternative of requiring repeated physician visits and cultures of all women who present with the symptoms of UTI is clearly inconvenient to the patient and physician and is unnecessarily expensive in the majority of cases. Therefore, there is an acute need for a method of treating routine UTI in an appropriate patient population that conserves health care resources while providing proper treatment to patients with more complicated conditions.

The diagnosis of UTI is complicated by the fact that there is a 48 to 72 hour lead time to obtain information from a urine culture on the type of pathogen and its antimicrobial susceptibility profile. The uncertainty about the proper course of treatment during this period can be reduced if comprehensive up-to-date data are available for the type of patient, regional location of the patient, and the timing of the infection.

Another aspect of increasing concern in UTI management is the growing microbial resistance to existing antibiotics. While there are other infectious diseases such as tuberculosis for which resistance is a serious problem, UTI is unique because of the large number of individuals affected and the difficulty of tracking resistance since cultures are generally not performed under the current treatment scheme. Since patients are generally treated on an outpatient basis, non-responsiveness to drug therapy could be due to drug resistance or to other factors such as non-compliance or misdiagnosis. Furthermore, drug resistant microbial strains can arise and spread very rapidly, but they are often localized within narrow geographical areas of socio-economic groups. Thus, even if there were more historical information available it may have little relevance to the current context due to rapid changes in drug resistance. Thus, drug resistance in any disease must be taken very seriously, but it cannot be monitored without a mechanism of collecting information, e.g., urine cultures.

### SUMMARY

Disclosed herein are systems and methods for significantly improving the accuracy of drug selection for patients diagnosed with, or symptomatic for, complicated and uncomplicated urinary tract infections (UTIs). In some embodiments, the systems and methods utilize real-time patient data linked to geographic distribution maps of resistance to different antimicrobial treatments for UTIs. The combination of real-time data and resistance maps allow for more accurate selection of appropriate UTI therapy for the patient.

In some embodiments, systems for selecting an antimicrobial for a patient presenting with a complicated or an uncomplicated urinary tract infection (UTI) are disclosed.

In some embodiments, the systems comprise at least one hardware processor that is programmed to: receive information indicative of a place of residence of the patient presenting with symptoms of the UTI; receive information indicating that the patient has a known history of previous uropathogen resistance to a previously administered antimicrobial; identify an algorithm to use to select an antimicrobial based on: (i) the information indicative of the place of residence of the patient presenting with symptoms of the UTI; (ii) the information indicating that the patient has a known history of previous uropathogen resistance to the previously administered antimicrobial; or (iii) both of (i) and (ii); select, using the algorithm, an antimicrobial that is predicted to be effective for treating the patient's UTI based on (i), (ii), or (iii); generate a report based on the selection of the antimicrobial that is predicted to be effective for treating the patient's UTI using the algorithm; and cause the report to be presented.

In some aspects of the system, the algorithm is configured to select an antimicrobial based on antimicrobial resistance associated with a geographic area with which the place of residence of the patient is associated. In some aspects of the system, the information indicative of the place of residence of the patient comprises a ZIP code. In some aspects of the system, the report comprises a map demarcated by ZIP code, the map depicting at least geographic areas associated with a plurality of ZIP codes including the ZIP code corresponding to the place of residence of the patient and one or more neighboring ZIP codes, and wherein each of the plurality of ZIP codes is coded to indicate a degree of resistance to the antimicrobial that is predicted to be effective for treating the second patient's UTI observed in residents of the geographic area corresponding to the respective ZIP code.

In some aspects of the system, the at least one hardware processor is further programmed to: receive information indicative of associations between geographic areas, including at least the geographic area associated with the plurality of ZIP codes, and resistance to a plurality of particular antimicrobials including at least the antimicrobial that is predicted to be effective for treating the second patient's UTI; and render the map based on the information indicative of associations between geographic areas and resistance to the plurality of particular antimicrobials.

In some aspects of the system, the at least one hardware processor is further programmed to: receive information indicating that the patient's UTI was resistant to the antimicrobial that is predicted to be effective for treating the patient's UTI; and update the information indicative of associations between geographic areas and resistance to a plurality of particular antimicrobials based on the information indicating that the patient's UTI was resistant to the antimicrobial that is predicted to be effective for treating the patient's UTI.

In some aspects of the system for selecting an antimicrobial for a patient presenting with a urinary tract infection (UTI), the at least one hardware processor is further programmed to: receive identifying information associated with the patient; and retrieve, from an electronic medical record system using the identifying information associated with the patient, the information indicative of the place of residence of the patient and the information indicating that the patient has a known history of previous uropathogen resistance to the first antimicrobial. In some aspects of the system, the system comprises the electronic medical record system.

In some aspects of the system for selecting an antimicrobial for a patient presenting with a urinary tract infection (UTI), the report comprises a map demarcated by ZIP code, the map depicting at least geographic areas associated with a plurality of ZIP codes including a ZIP code corresponding to the place of residence of the patient and one or more neighboring ZIP codes, and wherein each of the plurality of ZIP codes is coded to indicate a degree of resistance to the antimicrobial that is predicted to be effective for treating the patient's UTI observed in residents of the geographic area corresponding to the respective ZIP code.

In some aspects of the system for selecting an antimicrobial for a patient presenting with a urinary tract infection (UTI), the at least one hardware processor is programmed to: receive information indicative of a place of residence of a first patient presenting with symptoms of a UTI;

receive information indicating that the first patient has a known history of previous uropathogen resistance to a first antimicrobial; identify a first algorithm to use to select an antimicrobial based on the information indicating that the first patient has a known history of previous uropathogen resistance to the first antimicrobial; select, using the first algorithm, an antimicrobial that is predicted to be effective for treating the first patient's UTI based at least in part on the information indicating that the first patient has a known history of previous uropathogen resistance to the first antimicrobial; generate a first report based on the selection of the antimicrobial that is predicted to be effective for treating the first patient's UTI using the first algorithm, the first report comprising information indicating that the antimicrobial that is predicted to be effective based on a known history of previous uropathogen resistance; cause the first report to be presented; receive information indicative of a place of residence of a second patient presenting with symptoms of a UTI; identify a second algorithm to use to select an antimicrobial based on the place of residence of the second patient; select, using the second algorithm, an antimicrobial that is predicted to be effective for treating the second patient's UTI based at least in part on the information indicative of the place of residence of the second patient; generate a second report based on the selection of the antimicrobial that is predicted to be effective for treating the second patient's UTI using the second algorithm, the second report comprising information indicating that the antimicrobial is predicted to be effective based on the second patient's place of residence; cause the second report to be presented.

In some aspects of the system, the at least one hardware processor is further programmed to: receive information indicative of a place of residence of a third patient presenting with symptoms of a UTI; receive information indicating that the third patient previously received a prescription for a second antimicrobial; identify the first algorithm to use to select an antimicrobial based on the information indicating that the third patient previously received a prescription for the second antimicrobial; select, using the first algorithm, an antimicrobial that is predicted to be effective for treating the second patient's UTI based at least in part on the information indicating that the third patient previously received a prescription for the second antimicrobial; generate a third report based on the selection of the antimicrobial that is predicted to be effective for treating the third patient's UTI using the first algorithm, the first report comprising information indicating that the antimicrobial that is predicted to be effective based on the previous prescription for the second antimicrobial; and cause the third report to be presented.

In some aspects of the system, the second algorithm is configured to select an antimicrobial based on antimicrobial resistance associated with a geographic area with which the place of residence of the second patient is associated.

In some embodiments, methods for selecting an antimicrobial for a patient presenting with a complicated or an uncomplicated urinary tract infection (UTI) are disclosed.

In some aspects, the methods comprise: (a) receiving information indicative of a place of residence of the patient presenting with symptoms of the UTI; (b) receiving information indicating that the patient has a known history of previous uropathogen resistance to a previously administered antimicrobial; (c) identifying an algorithm to use to select an antimicrobial based on: (i) the information indicative of the place of residence of the patient presenting with symptoms of the UTI; (ii) the information indicating that the patient has a known history of previous uropathogen resistance to the previously administered antimicrobial; or (iii) both of (i) and (ii); (d) selecting, using the algorithm, an antimicrobial that is predicted to be effective for treating the patient's UTI based on (i), (ii), or (iii); (e) generating a report based on the selection of the antimicrobial that is predicted to be effective for treating the patient's UTI using the algorithm; and (f) causing the report to be presented.

In some aspects of the method, the information indicative of the place of residence of the patient comprises a ZIP code.

In some aspects of the method, the report comprises a map demarcated by ZIP code, the map depicting at least geographic areas associated with a plurality of ZIP codes including the ZIP code corresponding to the place of residence of the patient and one or more neighboring ZIP codes, and wherein each of the plurality of ZIP codes is coded to indicate a degree of resistance to the antimicrobial that is predicted to be effective for treating the patient's UTI observed in residents of the geographic area corresponding to the respective ZIP code.

In some aspects of the method, the method further comprises receiving information indicative of associations between geographic areas, including at least the geographic area associated with the plurality of ZIP codes, and resistance to a plurality of particular antimicrobials including at least the antimicrobial that is predicted to be effective for treating the second patient's UTI; and rendering the map based on the information indicative of associations between geographic areas and resistance to the plurality of particular antimicrobials.

In some aspects of the method, the method further comprises: receiving information indicating that the patient's UTI was resistant to the antimicrobial that is predicted to be effective for treating the patient's UTI; and updating the information indicative of associations between geographic areas and resistance to a plurality of particular antimicrobials based on the information indicating that the patient's UTI was resistant to the antimicrobial that is predicted to be effective for treating the patient's UTI.

In some aspects of the method, the method further comprises: receiving identifying information associated with the patient; and retrieving, from an electronic medical record system using the identifying information associated with the patient, the information indicative of the place of residence of the patient and the information indicating that the patient has a known history of previous uropathogen resistance to the previously administered antimicrobial.

In some aspects of the method, the report comprises a map demarcated by ZIP code, the map depicting at least geographic areas associated with a plurality of ZIP codes including a ZIP code corresponding to the place of residence of the patient and one or more neighboring ZIP codes, and wherein each of the plurality of ZIP codes is coded to indicate a degree of resistance to the antimicrobial that is predicted to be effective for treating the patient's UTI observed in residents of the geographic area corresponding to the respective ZIP code.

Is some aspects of the method, the method comprises: receiving information indicative of a place of residence of a first patient presenting with symptoms of a UTI; receiving information indicating that the first patient has a known history of previous uropathogen resistance to a first antimicrobial; identifying a first algorithm to use to select an antimicrobial based on the information indicating that the first patient has a known history of previous uropathogen resistance to the first antimicrobial; selecting, using the first algorithm, an antimicrobial that is predicted to be effective for treating the first patient's UTI based at least in part on the information indicating that the first patient has a known history of previous uropathogen resistance to the first antimicrobial; generating a first report based on the selection of the antimicrobial that is predicted to be effective for treating the first patient's UTI using the first algorithm, the first report comprising information indicating that the antimicrobial that is predicted to be effective based on a known history of previous uropathogen resistance; causing the first report to be presented; receiving information indicative of a place of residence of a second patient presenting with symptoms of a UTI; identifying a second algorithm to use to select an antimicrobial based on the place of residence of the second patient; selecting, using the second algorithm, an antimicrobial that is predicted to be effective for treating the second patient's UTI based at least in part on the information indicative of the place of residence of the second patient; generating a second report based on the selection of the antimicrobial that is predicted to be effective for treating the second patient's UTI using the second algorithm, the second report comprising information indicating that the antimicrobial is predicted to be effective based on the second patient's place of residence; causing the second report to be presented.

In some aspects of the method, the method further comprises: receiving information indicative of a place of residence of a third patient presenting with symptoms of a UTI; receiving information indicating that the third patient previously received a prescription for a second antimicrobial; identifying the first algorithm to use to select an antimicrobial based on the information indicating that the third patient previously received a prescription for the second antimicrobial; selecting, using the first algorithm, an antimicrobial that is predicted to be effective for treating the second patient's UTI based at least in part on the information indicating that the third patient previously received a prescription for the second antimicrobial; generating a third report based on the selection of the antimicrobial that is predicted to be effective for treating the third patient's UTI using the first algorithm, the first report comprising information indicating that the antimicrobial that is predicted to be effective based on the previous prescription for the second antimicrobial; and causing the third report to be presented.

In some aspects of the method, the second algorithm is configured to select an antimicrobial based on antimicrobial resistance associated with a geographic area with which the place of residence of the second patient is associated.

In any of the above aspects of the system, the patient's UTI may be a complicated UTI.

In any of the above aspects of the method, the patient's UTI may be a complicated UTI.

### BRIEF DESCRIPTION OF THE DRAWINGS

FIG. 1. Graph showing ROC curves for predicting negative culture by positive culture cutoff. Data lines from left to right are: 100,000; 10,000; and 1000.

FIG. 2. City of Chicago maps by ZIP codes with resistance to (A) trimethoprim-sulfamethoxazole, (B) nitrofurantoin, and (C) ciprofloxacin.

FIG. 3. Table showing univariate and multivariable analysis of selected predictors of resistance to (A) SXT (trimethoprim-sulfamethoxazole), (B) NIT (nitrofurantoin), and (C) CIP (ciprofloxacin).

FIG. 4. Algorithms using (A) previous uropathogen resistance to an antimicrobial and prior prescription data for patients with prior microbiologic data; and (B) ZIP code data only for patients without any prior microbiologic data. CIP, ciprofloxacin; NIT, nitrofurantoin; SXT, trimethoprim-sulfamethoxazole.

FIG. 5. Table summarizing for patients with prior microbiolgic data: (A) antimicrobial choice by provider at time of diagnosis with susceptibilities; and (B) antimicrobial choice based on past resistance and prior prescriptions only with susceptibilities; (C) breakdown of susceptibilities by antimicrobial choice when choice based on previous resistance and prior prescriptions in those with prior microbiologic data. For patients without prior microbial data: (D) provider choices of empirical therapy; (E) antimicrobial choice based on ZIP code alone; (F) evaluation of groups (D) and (E).

FIG. 6. Shows UTI antimicrobial resistance patterns by ZIP code in the city of Chicago. (A) City of Chicago maps by ZIP code with resistance for CIP, SXT, and NIT for complicated compared to uncomplicated UTI (column 1 vs. 2); resistance of CF-1 and CF-3 for complicated UTI only (column 3). (B) Comparisons of uncomplicated and complicated UTI populations by antimicrobial (CIP, NIT, SXT).

FIG. 7. Table summarizing significant predictors in multivariable analysis by antimicrobial of interest. SXT—Trimethoprim-Sulfamethoxazole, CIP—Ciprofloxacin, NIT—Nitrofurantoin, CF-1-First Generation Cephalosporin, CF-3-Third Generation Cephalosporin, t-Antimicrobial of interest for each column is regardless of which uropathogen organism was identified on past or current urine cultures.

FIG. 8. Shows outpatient treatment algorithms for complicated UTI. Algorithms for outpatients presenting with complicated UTI without prior urine culture resistance data (A) or with prior urine culture resistance data (B). For patients without prior urine culture resistance data, antimicrobial resistance data based on ZIP code (presumed location of UTI acquisition) was used (A). SXT—Trimethoprim-sulfamethoxazole. CIP—Ciprofloxacin. NIT—Nitrofurantoin. CF1-First-generation cephalosporin. CF3-Third-generation cephalosporin.

FIG. 9. In patients with complicated UTIs without prior microbiologic data: (A) antimicrobial choice by provider at time of diagnosis with susceptibilities and (B) antimicrobial choice based on patient place of residence (ZIP code) from data derived from complicated UTIs only with susceptibilities. (C) antimicrobial choice based on patient place of residence (ZIP code) from data derived from uncomplicated UTIs only with susceptibilities. *-p=0.026. †-p=0.002. SXT—Trimethoprim-sulfamethoxazole. CIP—Ciprofloxacin. NIT—Nitrofurantoin. CF-1-First Generation Cephalosporin. CF-3-Third Generation Cephalosporin.

FIG. 10. Shows an example of a system that can be used to select an antimicrobial for a patient presenting with a UTI in accordance with some embodiments of the disclosed subject matter.

FIG. 11. Shows an example of hardware that can be used to implement a computing device and/or a server in accordance with some embodiments of the disclosed subject matter.

FIG. 12. Shows an example of a process for training a system for predicting an effective antimicrobial for treating a patient's UTI based on residence information and medical history in accordance with some embodiments of the disclosed subject matter.

FIG. 13. Shows an example of a process for predicting an effective antimicrobial for treating a patient's UTI based on residence information and medical history in accordance with some embodiments of the disclosed subject matter.

### DETAILED DESCRIPTION

Approximately half of all women will have a urinary tract infection (UTI) in their lifetimes and more than a third will have recurrent UTIs.¹ UTI represents a large burden on our health care system in terms of patient prevalence as well as financial impact (approximately 2.6 billion USD in 2010).²⁻⁴ Most women have uncomplicated UTIs. Current recommendations include treating empirically with antimicrobials without first confirming antimicrobial susceptibilities.^{5, 6} In the United States, recommendations for antimicrobial selection include nitrofurantoin (NIT) or trimethoprim-sulfamethoxazole (SXT). If either of these are not a viable option, choose a fluoroquinolone, such as ciprofloxacin (CIP).^{5, 6} Use of NIT for uncomplicated UTIs has increased over the past decades as a first line agent.⁷

Prior antimicrobial resistance can be helpful in guiding directed antimicrobial choice for patients with both complicated and uncomplicated UTIs, and many researchers suggest recent prescriptions may be useful in predicting resistance.⁸⁻¹¹ Other studies have shown that antimicrobial resistance is often geographically distributed throughout the community for UTIs and other infections.¹²⁻¹⁴ We are not aware of any studies using a patient's place of residence to predict antimicrobial resistance for uncomplicated UTIs.

We hypothesized that algorithms using patients' prior culture resistance, antimicrobial prescription history and place of residence (e.g., ZIP code) can guide antimicrobial therapy in patients with either complicated or uncomplicated UTIs and outperform current guidelines.

We demonstrate herein that for women with no prior microbiologic data available, an algorithm using residence information (e.g., ZIP code data) can outperform these guidelines and real-world practices for uncomplicated UTIs. A separate algorithm using residence information (e.g., ZIP code data), outperforms current guidelines for women with complicated UTIs and having no prior microbiologic data available.

Thus, in some embodiments, the systems and methods disclosed herein are utilized to collect urine culture data on women presenting with uncomplicated UTIs to evaluate for ZIP codes with high resistance rates to the above antimicrobials. In some embodiments, the systems and methods are connected to the electronic medical records (EMR) and will function to prospectively collect data on urine culture resistance patterns and associated ZIP codes. This data will be used to determine areas of high and low probability of antimicrobial resistance. Accordingly, when a new patient is being seen for treatment of a presumed uncomplicated UTI, the aggregated data would then be used to choose an antimicrobial most likely to be effective for this patient. In some embodiments, the patient is suffering from a UTI, and in some embodiments, the patient is suffering from an uncomplicated UTI.

In some embodiments, the systems and methods disclosed herein are utilized to collect urine culture data on women presenting with complicated UTIs to evaluate for residence locations (e.g., ZIP codes) with high resistance rates to the above antimicrobials. In some embodiments, the systems and methods are connected to the electronic medical records (EMR) and will function to prospectively collect data on urine culture resistance patterns and associated residence locations (e.g., ZIP codes). This data will be used to determine areas of high and low probability of antimicrobial resistance. Accordingly, when a new patient is being seen for treatment of a presumed complicated UTI, the aggregated data would then be used to choose an antimicrobial most likely to be effective for this patient. In some embodiments, the patient is suffering from a UTI, and in some embodiments, the patient is suffering from a complicated UTI.

As used herein the term “ZIP code” refers to a 5 or 9 digit number that identifies a particular postal delivery area. However, the systems and methods described herein are not limited by the term “ZIP code.” Rather, this term is used as a convenient means to identify a geographic location, such as place of residence or area of residence, within the United States. In the disclosed methods, it should be understand that other manners of identifying and specifying a particular geographic location or a geographic region (e.g., a place of residence) can be used. For example, a GPS location, a patient's address, a regionally identified zone, township, range, a defined area based on square mileage, a defined area based on population, a defined area based on population per acre, etc. may be employed to identify a subject's place of residence, or an area of residence.

As described herein and as shown in the Examples below, previous antimicrobial resistance is a predictor of future antimicrobial resistance for UTIs. In addition, patient place of residence is an important predictor of antimicrobial resistance in subjects without prior microbiologic data available.

With respect to complicated versus uncomplicated UTIs, there is no correlation between the uncomplicated and complicated ZIP code maps. Thus, the status of the UTI (complicated versus uncomplicated) is important, as algorithms and maps based on one cannot usefully predict resistance in the other group. However, the exemplary systems and methods describe below are useful for developing maps and algorithms for the prediction of antimicrobial treatment for either complicated or uncomplicated UTIs.

Systems

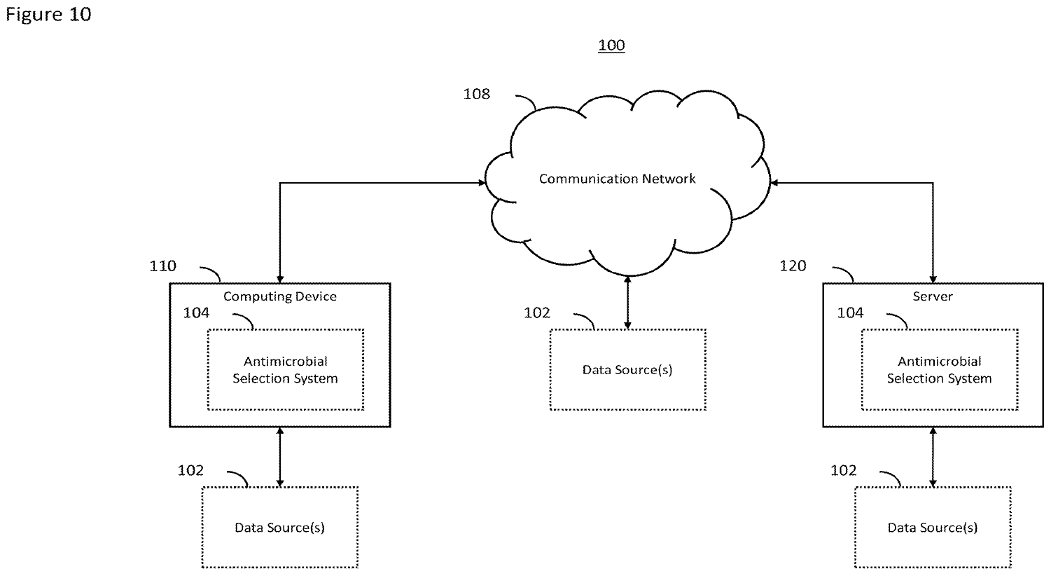

FIG. 10 shows an example 100 of a system that can be used to select an antimicrobial for a patient presenting with a UTI in accordance with some embodiments of the disclosed subject matter. As shown in FIG. 10, a computing device 110 can receive data related to the patient (e.g., age, race, sex, patient's place of residence (e.g., ZIP code, address, GPS coordinates, etc.), medical history, etc.) from a data source 102 that stores such data, and/or from an input device (e.g., input provided via a mouse, a keyboard, a touchscreen, etc., by a medical provider or associated personnel, by the patient). In some embodiments, computing device 110 can execute at least a portion of an antimicrobial selection system 104 to predict an effective antimicrobial for treating the patient's UTI (e.g., an antimicrobial to which the UTI is unlikely to be resistant) based on data received from data source 102 and/or the input device.

Additionally or alternatively, in some embodiments, computing device 110 can communicate information about data received from data source 102 to a server 120 over a communication network 108 and/or server 120 can receive data from data source 102 (e.g., directly and/or using communication network 108), which can execute at least a portion of antimicrobial selection system 104 to predict an effective antimicrobial for treating the patient's UTI (e.g., an antimicrobial to which the UTI is unlikely to be resistant) based on data received from data source 102 and/or the input device. In such embodiments, server 120 can return information to computing device 110 (and/or any other suitable computing device) indicative of an antimicrobial predicted to be effective for treating the patient's UTI.

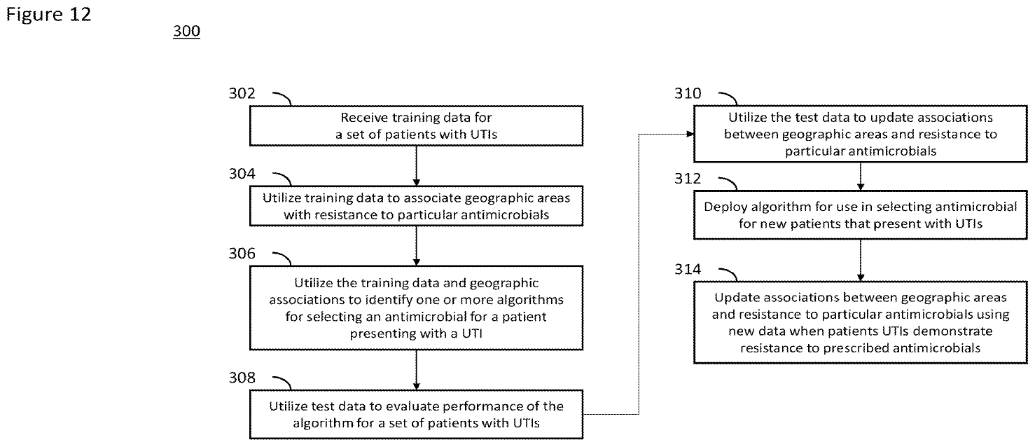

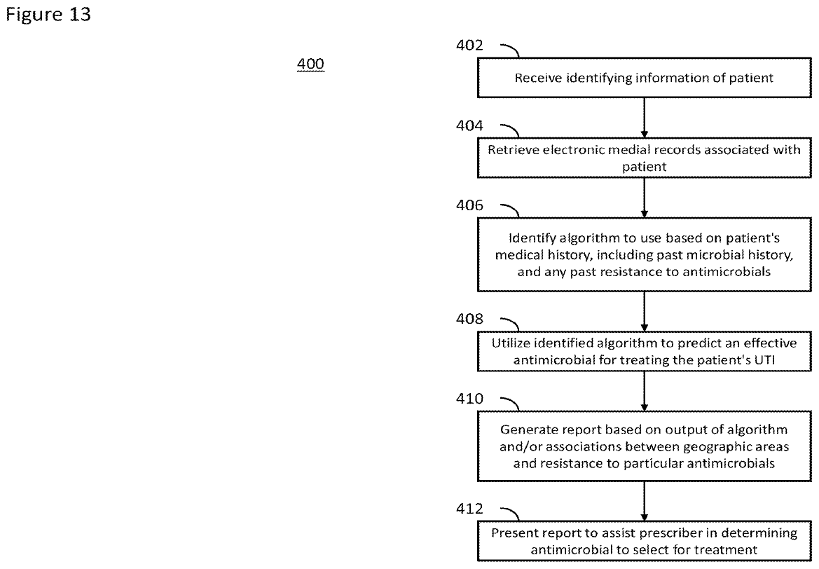

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, etc. As described below in connection with FIGS. 12 and 13, in some embodiments, computing device 110 and/or server 120 can receive training data (e.g., data associated with patients for whom susceptibility and/or resistance to one or more antimicrobials is known, for example, as described in connection with Examples 1 to 3) from one or more data sources (e.g., data source 102), and can format the data for use in identifying associations between antimicrobial resistance and geographical location or areas where the patients reside, and/or for use in identifying associations between other characteristics of the patients and antimicrobial resistance. In some embodiments, antimicrobial selection system 104 can use the training data to develop algorithms to predict an effective antimicrobial for treating a patient's UTI.

In some embodiments, antimicrobial selection system 104 can receive data associated with a patient presenting with a UTI for whom susceptibility and/or resistance to one or more antimicrobials is unknown from one or more sources of data (e.g., data source 102), and can format the data for input use by one or more algorithms to predict an effective antimicrobial for treating a patient's UTI. In some embodiments, antimicrobial selection system 104 can identify an antimicrobial that is predicted to be effective for treating the patient's UTI based on the data, and can present the results for a user (e.g., a physician, a nurse, a pharmacist, etc.) to utilize in making a treatment decision.

In some embodiments, data source 102 can be any suitable source or sources of data. For example, data source 102 can be an electronic medical records system and/or a database associated with an electronic medical records system. As another example, data source 102 can be a computing device used to collect data about the patient (e.g., a smartphone, a tablet computer, a desktop computer, a laptop computer, etc., that presents a user interface that can be used to enter data associated with the patient). As yet another example, data source 102 can be an input device (e.g., a keyboard, a touchscreen, a paper form) that facilitates manual data entry by a user. As still another example, data source 102 can be data stored in memory of computing device 110 and/or server 120 using any suitable format, such as using a database, a spreadsheet, a document with data entered using a comma separated value (CSV format), and/or any other suitable format.

In some embodiments, data source 102 can be local to computing device 110. For example, data source 102 can be incorporated with computing device 110 (e.g., using memory associated with computing device). As another example, data source 102 can be connected to computing device 110 by one or more cables, a direct wireless link, etc. Additionally or alternatively, in some embodiments, data source 102 can be located locally and/or remotely from computing device 110, and send data to computing device 110 (and/or server 120) via a communication network (e.g., communication network 108).

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, 5G NR, etc.), a wired network, etc. In some embodiments, communication network 2108 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, etc.

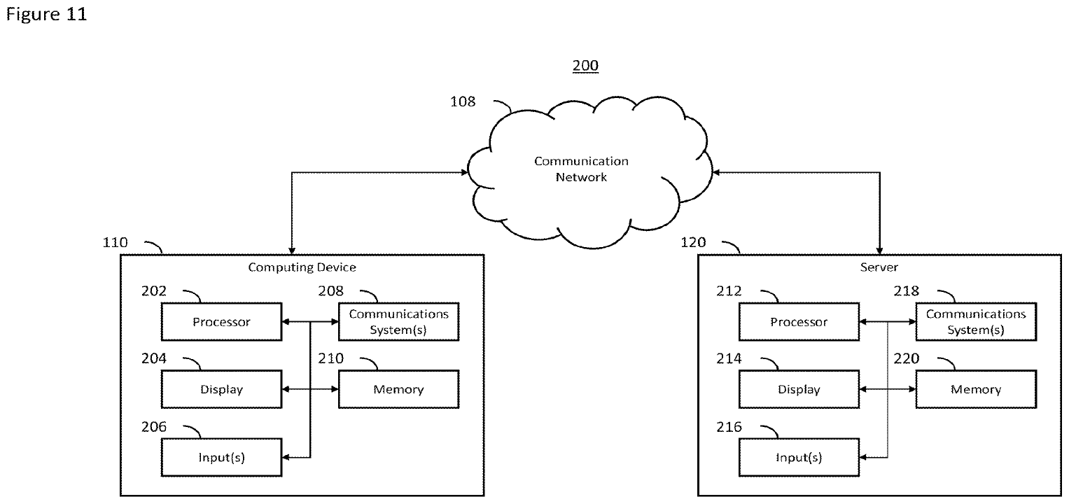

FIG. 11 shows an example 200 of hardware that can be used to implement computing device 110, and/or server 120 in accordance with some embodiments of the disclosed subject matter. As shown in FIG. 11, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 204 can include any suitable display devices, such as a computer monitor, a touchscreen, a television, etc. In some embodiments, inputs 206 can include any suitable input devices that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to present content (e.g., user interfaces, graphics, tables, reports, etc.), receive content from server 120, transmit information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an MCU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to transmit information and/or content (e.g., a user interface, graphs, tables, reports, etc.) to one or more computing devices 110, receive information and/or content from one or more computing devices 110, receive instructions from one or more devices (e.g., a personal computer, a laptop computer, a tablet computer, a smartphone, etc.), etc.

FIG. 12 shows an example 300 of a process for training a system for predicting an effective antimicrobial for treating a patient's UTI based on residence information and medical history in accordance with some embodiments of the disclosed subject matter. As shown in FIG. 12, at 302, process 300 can receive training data for a set of patients that were treated for UTIs, and for which resistance and/or susceptibility information of the UTI to one or more antimicrobials is known. For example, the training data can include data described above in connection with one or more of Examples 1 to 3. In some embodiments, the training data can include various data associated with the patient, such as uropathogen resistance (defined as resistant or intermediate susceptibility) to one or more antimicrobials (e.g., SXT, NIT, CIP, CF1, CF3, TMP-SMX, and/or any other suitable antimicrobial), patient demographics (e.g., age, race patient's place of residence (e.g., ZIP code, address, GPS coordinates, etc.), antimicrobial prescriptions within the past two years, diagnosis codes associated with current and past encounters, past urine culture results and department to which they presented, and/or any other suitable data.

At 304, process 300 can include utilizing the training data to associate geographic areas (e.g., defined based on postal codes such as ZIP codes, or other using other geographic descriptors, such as census tracts) with resistance to particular types of antimicrobials based on the training data. In some embodiments, process 300 can use any suitable technique or combination of techniques to determine an association between a particular geographic area and resistance to a particular type of antimicrobial. For example, one or more techniques described in connection with Examples 2 to 3 can be used to identify associations between particular geographic areas and resistance to a particular type of antimicrobial.

At 306, process 300 can include utilizing the training data and the geographic associations to identify one or more algorithms for selecting an antimicrobial for a patient presenting with a UTI. In some embodiments, any suitable technique or combination of techniques can be used to identify one or more algorithms for selecting an antimicrobial that is predicted to be effective for treating a patient's UTI based on data associated with the patient. For example, one or more techniques described above in connection with Examples 2 to 3 can be used to identify one or more algorithms for selecting.

At 308, process 300 can utilize test data to evaluate performance of one or more of the algorithms identified at 306 for a set of patients that were treated for UTIs, and for which resistance and/or susceptibility information of the UTI to one or more antimicrobials is known. For example, the test data can include data described above in connection with one or more of Examples 1 to 3. In a more particular example, the test data can include data that represents patients with UTIs that occurred later in time, such that the training data precedes the test data in time. As described above in connection with Examples 1 to 3, the test data can be used to evaluate the predictive power of the algorithm(s), and evaluate the performance of the algorithm in selecting an antimicrobial that would have likely been effective in treating the UTI (e.g., based on the results of a culture) with respect to the selection made by the prescriber.

At 310, process 300 can utilize the test data to update associations between geographic areas and resistance to particular antimicrobials. In some embodiments, after verifying the performance of the one or more algorithms using the test data, the test data can be used to update the associations between the geographic regions and resistance to particular antimicrobials. For example, techniques described above in connection with 304 and/or Examples 1 to 3 can be used to account for the test data in the associations between geographic areas and particular antimicrobials. In such an example, after excluding the test data from the associations prior to testing (e.g., to avoid fitting the algorithm to the test data), the test data can be used to update the geographic associations to antimicrobial resistance to make the associations more robust.

At 312, process 300 can deploy the one or more algorithms for use in selecting an antimicrobial for new patients that present with UTIs. For example, as described below in connection with FIG. 13, the one or more algorithms can be used to assist a medical practitioner in identifying an antimicrobial that is most likely to be effective to treat a patients UTI based on the patient's residence location information and/or previous uropathogen resistance.

At 314, process 300 can update associations between geographic areas and resistance to particular antimicrobials using new data that is generated when patients' UTIs demonstrate resistance to prescribed antimicrobials. In some embodiments, when a bacterial culture is performed, a UTI diagnosis is confirmed (e.g., via the culture), and resistance to one or more antimicrobials is indicated by the culture, the degree of resistance demonstrated to one or more antimicrobials and the patient's residence location information (e.g., abstracted to a broader area, such as a ZIP code) can be added as a new data point(s) to the data used to associate geographic areas and resistance to particular antimicrobials. A bacterial culture can be performed for various reasons. For example, if a patient has been diagnosed with and/or is symptomatic for a UTI is prescribed an antimicrobial (e.g., with the assistance of one or more of the algorithms deployed at 312), and the UTI appears to be resistant to the antimicrobial, a culture can be performed to confirm the diagnosis of a UTI and/or to confirm whether the UTI is resistant to the prescribed antimicrobial. As another example, if any clinical features or other factors suggest a complicated infection, a bacterial culture can be performed prior to prescribing of a particular antimicrobial therapy is started.

In some embodiments, process 300 can update the associations between the geographic regions and resistance to particular antimicrobials at regular and/or irregular intervals, and using any suitable technique or combination of techniques. For example, process 300 can use one or more techniques described above in connection with 304 and/or Examples 1 to 3 to account for new data in the associations between geographic areas and particular antimicrobials. As another example, process 300 can update the associations after a particular period of time has elapsed since a last update (e.g., after a day, a week, two weeks, one month, etc.) has elapsed. As yet another example, process 300 can update the associations after a particular amount of new data has been collected (e.g., new data for a particular geographic area, or new data across all geographic areas in a particular region, such as a neighborhood, city, metropolitan area, state, country, etc.).

FIG. 13 shows an example 400 of a process for predicting an effective antimicrobial for treating a patient's UTI based on residence information and medical history in accordance with some embodiments of the disclosed subject matter. As shown in FIG. 13, at 402, process 400 can receive identifying information associated with a patient. For example, such identifying information can be a patient identifier that uniquely identifies the patient in an electronic medical record system. As another example, such identifying information can be a username and/or password that the patient has established to access the electronic medical record system. As yet another example, such identifying information can be biometric information association with the patient, such as a fingerprint, palm print, retina scan, etc.

At 404, process 400 can retrieve electronic medical records association with the patient based on the identifying information of the patient received at 402. In some embodiments, process 400 can retrieve the electronic medical records from any suitable device or storage location, such as an electronic medical records system which can be local to a device executing at least a portion of process 400 and/or remote from the device executing at least a portion of process 400.

Additionally or alternatively, at 404, in some embodiments, the patient can be prompted to provide demographic information (e.g., patient's place of residence, including at least ZIP code, age, race, etc.) medical history information and/or information about symptoms currently being experienced, including an indication of whether the patient has previously been diagnosed with a UTI and/or been prescribed one or more antimicrobials, whether one or more factors are present that indicate that the UTI is a complicated UTI, etc. In some embodiments, 2404 can be omitted, such as in examples in which a patient's information is not stored in an electronic medical record system.

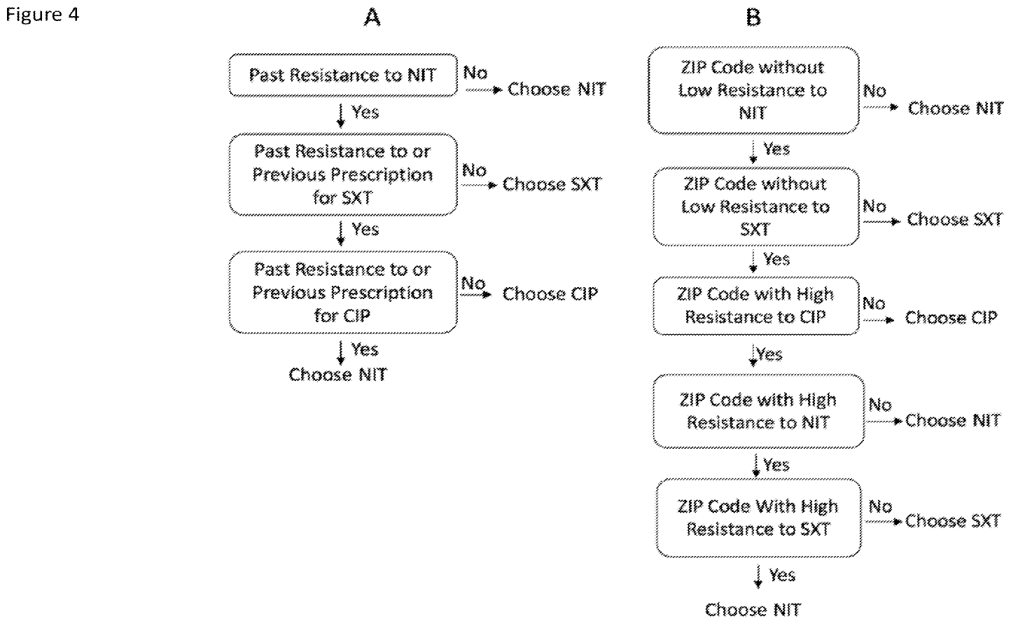

At 406, process 400 can identify an algorithm to use based on the patient's medical history, including past microbial history and/or any past uropathogen resistance. In some embodiments, process 400 can use any suitable technique or combination of techniques to identify an algorithm to use, such as whether the patient's medical history and/or symptoms indicates that the UTI is an uncomplicated UTI or a complicated UTI. In some embodiments, process 400 can select an algorithm described above in connection with FIGS. 4, and/or 8, and in connection with Examples 2 to 3 based on the patient's medical history and/or symptoms.

At 408, process 400 can utilize the identified algorithm to predict an effective antimicrobial for treating the patient's UTI based on the patient's residence information and/or medical history. In some embodiments, process 400 can predict an effective antimicrobial as described above in connection with FIGS. 4, and/or 8, and in connection with Examples 2 to 3 based on the patient's medical history and/or symptoms.

At 410, process 400 can generate a report based on the output of the algorithm and/or based on associations between geographic areas and resistance to particular antimicrobials. In some embodiments, the report can include an indication of the antimicrobial selected. Additionally, in some embodiments, the report can include graphical information that a user (e.g., a medical practitioner making a treatment decision) can use to determine the appropriateness of the selection based on the underlying data. For example, the report can include a map that is coded by ZIP code to present resistance to the selected antibacterial and/or one or more other antimicrobials, with the patient's place of residence plotted on the map to provide the user with context that the user can use to evaluate a likelihood that the algorithm made an appropriate selection.

At 412, process 400 can cause the report to be presented to a user to assist the user in determining an antimicrobial to select for treatment of the patients UTI. For example, process 400 can cause the report to be presented using a graphical user interface (e.g., of computing device 110). As another example, process 400 can cause the report to be printed for review by the user.

In some embodiments, the report can be used to provide context to the user to confirm the selection by the algorithm, or determine that further investigation is warranted. For example, if the report indicates that the selection of the antimicrobial was made based on the ZIP code associated with the patient's place of residence, the report can include a map indicating that the ZIP code in which the patient's place of residence is located does not exhibit resistance to the selected antimicrobial. In a more particular example, the patient's place of residence may be near a border of a neighboring ZIP code that does exhibit high resistance to the antimicrobial. This may cause the user to conclude that the algorithm did not have sufficient data to determine an appropriate selection, and may determine that further investigation is warranted before prescribing the selected antimicrobial (e.g., by investigating the data in graphical form, by ordering a culture, etc.). As another example, if the report indicates that the selection of the antimicrobial was made based on a history of previous uropathogen resistance and/or prior prescription data, the report can include a map(s) indicating resistance to one or more antimicrobials, if any, by ZIP code, including a ZIP code in which the patient's place of residence is located and one or more neighboring ZIP codes. In a more particular example, the patient's place of residence may be located within a ZIP code, or near a border of a neighboring ZIP code, that exhibits high resistance to an antimicrobial that was selected based on the patient's history of previous uropathogen resistance and/or prior prescription data. This may cause the user to conclude that the UTI may have a relatively high likelihood of being resistant to the antimicrobial selected by the algorithm, and may determine that further investigation is warranted before prescribing the selected antimicrobial (e.g., by investigating the data in graphical form, by ordering a culture, etc.).

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be understood that the above described steps of the processes of FIGS. 4, 8, 12 and 13 can be executed or performed in any suitable order or sequence not limited to the order and sequence shown and described in the figures. Also, some of the above steps of the processes of FIGS. 4, 8, 12 and 13 can be executed or performed substantially simultaneously where appropriate or in parallel to reduce latency and processing times.

Applications

Exemplary, non-limiting applications of the systems and methods disclosed herein include the following.

The systems and methods disclosed herein could be an add-on in clinic electronic medical records (EMR) (as well as a stand-alone application) to aid in treatment of new patients with complicated or uncomplicated UTIs;

The systems and methods disclosed herein could be used as an add-on in EMRs for telemedicine providers;

The systems and methods disclosed herein could be used as a stand-alone for a patient facing application for self-diagnosis and treatment of uncomplicated UTIs.

Advantages

Exemplary, non-limiting advantages of the systems and methods disclosed herein include the following.

Current options are to treat empirically using static, historic guidelines or waiting for a culture to return with susceptibility results.

In contrast, the systems and methods disclosed herein integrate real-time data linked to geographic distribution to significantly improve the accuracy of drug selection for new patients with complicated or uncomplicated UTIs. This provides added confidence (in comparison to guidelines) to those who choose to treat immediately without waiting for culture results. The systems and methods disclosed herein provide for superior antimicrobial stewardship (better coverages without using broader spectrum drugs). Patient satisfaction and health are also increased due to accurate treatment and reducing or eliminating second visits due to inaccurate therapy for an infection.

### EXPERIMENTAL EXAMPLES

The following Examples are illustrative and should not be interpreted to limit the scope of the claimed subject matter.

### Example 1: Prior Culture Data Alone can Significantly Reduce Unnecessary Antimicrobial Therapy for Uncomplicated Urinary Tract Infections

A. Introduction and Objectives

Recommendations for treating an uncomplicated urinary tract infection (UTI) are to treat with antibiotics without performing a urine culture. However, only approximately 50% of women with a symptom of a UTI are ultimately found to have a positive culture (PC), resulting in unnecessary antibiotic use. Utilizing symptomatology and Urinalysis (UA) results can increase odds of correctly identifying PC, but data is limited on how prior cultures can affect the likelihood of a PC. How can prior culture data tell us who does not need antibiotics?

B. Methods

The Northwestern Enterprise Data Warehouse was queried for women (age>18) submitting urine cultures as outpatients in the Northwestern Healthcare System from 2011 to 2017. Diagnosis codes were used to exclude those without an uncomplicated UTI. Demographic data, healthcare use data, diagnosis codes, UA and urine culture results were collected. Cutoffs for PCs were evaluated at 10³, 10⁴ and 10⁵ cfu/mL. Univariate and multivariable regression models determined likelihood ratios (LR) for predicting positive urine culture (10⁴ cfu/mL).

C. Results

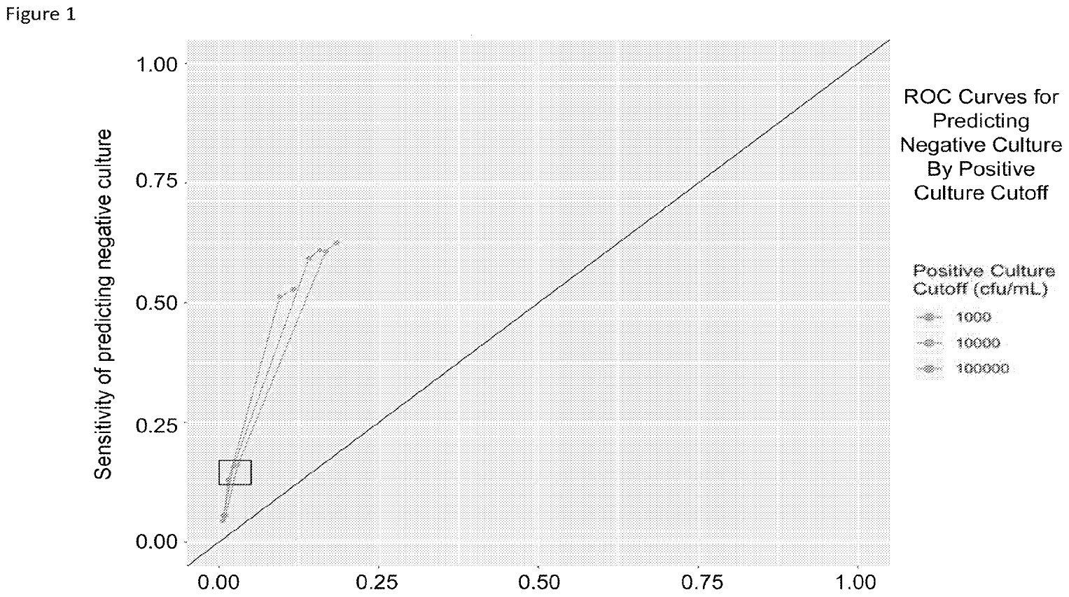

In the 20,759 patients with uncomplicated UTIs, for the cutoffs 10³, 10⁴ and 10⁵ cfu/mL, 11,488 (55.3%), 10,451 (50.3%) and 6,996 (33.7%) had PCs, respectively. Factors associated with decreased likelihood of PC: prior negative culture(s) (LR+0.651 [95% CI 0.625-0.679] p<0.001), having a PC within the past month (LR+0.726 [95% CI 0.678-0.777] p<0.001) and having both negative leukocyte esterase (LE) and nitrite on UA (LR+0.263 [95% CI 0.243-0.285] p<0.001). On multivariable analysis, negative prior culture with negative LE and negative nitrite on UA has a decreased likelihood of PC (LR+0.33 [95% CI 0.24-0.45] p<0.001) and is highly specific for predicting a negative culture (Sensitivity 0.157 [95% CI 0.156-0.157], Specificity 0.978 [95% CI 0.977-0.978], Predictive Value 0.891 [95% CI 0.891-0.892]). This point is inscribed on the operator curve in FIG. 1.

D. Conclusions

In patients presenting with symptoms of an uncomplicated UTI, the presence of a prior negative culture with a negative LE and nitrite on UA means the patient is very unlikely to have a PC. These criteria will falsely identify only 2.2% of patients with a PC as having a negative culture. This data can be used to determine who may benefit from further work up and who from immediate treatment.

### Example 2: Algorithms Using Previous Resistance, Prior Antimicrobial Prescriptions, and Patient Place of Residence Enhance Empirical Therapy for Women with Uncomplicated Urinary Tract Infections

A. Objective

The objective of this example was to evaluate how previous antimicrobial resistance, prior prescription data, and patient place of residence (ZIP code) can guide empirical therapy for uncomplicated urinary tract infections (UTI). Guidelines recommend empirical antimicrobial selection for women with symptoms of uncomplicated UTIs, most commonly trimethoprim-sulfamethoxazole (SXT), nitrofurantoin (NIT), or ciprofloxacin (CIP). Previous antimicrobial resistance and prior prescription data are potential predictors of resistance in subsequent urine cultures for UTIs. Also, there is evidence of geographic clustering of antimicrobial resistance for UTIs.

B. Materials and Methods

1) Study Design, Setting and Patients

The patient selection methods were replicated from a previous study that showed predictors of culture negativity in the same patients.¹⁵ This is a retrospective cross-sectional study of non-pregnant women aged 18 or older that submitted urine cultures as outpatients from 2011-2018 at any Northwestern Medicine site, which encompasses the greater Chicago area. Data were obtained from the Northwestern Medicine Enterprise Data Warehouse, which is a data integration warehouse designed for research and clinical operations. Patients were included for analysis only if the encounter at which the urine culture was collected had an ICD 9 (599.0 & 595) or 10 (N39.0 & N30) diagnosis code for symptomatic UTI. Patients were excluded if they were linked to any ICD 9 or 10 diagnosis code that would have been associated with a complicated UTI.¹⁶ The correlation between the diagnostic codes and the clinical presentation was confirmed by chart review of 300 cases; only 12 (4%) did not have documented UTI symptoms in the encounter note.¹⁵ Patients were included exactly once based on their most recent cultures, and all prior culture data were used as covariates. Cultures were included in analysis if positive (identified uropathogen≥10³ colony forming units per milliliter (CFU/mL)) and antimicrobial susceptibilities reported for the dominant species. The colony count cutoff of ≥10³ CFU/mL was used as it has been noted that low thresholds have appropriate diagnostic value for women with symptoms of a UTI.^{5, 17, 18} The Northwestern Medicine Institutional Review Board approved this study (STU00207763).

All specimen collection and testing were done uniformly in accordance with Northwestern Medicine Guidelines. Isolation, colony count and identification testing were performed using Vitek 2 cards (bioMérieux).

2) Training Population

For the 2011-2017 patients, outcomes of interest were uropathogen resistance (defined as resistant or intermediate susceptibility) to SXT, NIT and CIP. Covariates were patient demographics (age, race and patient's place of residence (ZIP code)) antimicrobial prescriptions within the past two years, diagnosis codes associated with current and past encounters, past urine culture results and department to which they presented. Risk factors were compared between patients who had resistant and sensitive cultures using two-sample t-tests (continuous variables) and chi-squared tests (categorical variables). Multivariate logistic regression models were used to investigate the association between risk factors and resistant cultures. Odds ratios (OR) were derived from these models to determine the probability of having a resistant culture with those factors.

Algorithms were then created using the risk factors found to be most predictive of a resistant culture in the analysis described above.

3) Testing Population

The algorithms developed using the above-described cohort were then tested on the 2018 cohort. The overall success of these algorithms (proportion with a chosen antimicrobial that had a culture susceptible to that drug) was determined and compared to provider-chosen therapy and guideline chosen therapy. All analyses were done using R (version 3.5.2).

C. Results

1) Training Population

Over the study period, 9,455 women with diagnoses of uncomplicated UTIs and positive urine cultures (≥10³ CFU/mL) with antimicrobial susceptibilities available were identified. The mean age was 48.7 years; 1,978 (20.9%) had a prior positive culture; 6,660 (70.4%) were white. The most common departments at which the cultures were obtained were the Emergency Department or Immediate Care (40%), Internal Medicine (38%) and Obstetrics and Gynecology (10%). The most common uropathogen was Escherichia coli (74%) followed by Group B Streptococci (6%), Klebsiella pneumoniae (6%) and Enterococcus spp. (3%).

Of the 9,455 cultures, 8,886 (94.0%), 9,397 (99.4%) and 9,355 (98.9%) had susceptibilities reported to SXT, NIT and CIP, respectively. Overall prevalence of resistance for SXT, NIT and CIP were 19.4%, 12.1% and 10.3%, respectively. Resistance was stable throughout the study period for all three antimicrobials.

2) Antimicrobial Resistance by ZIP Code

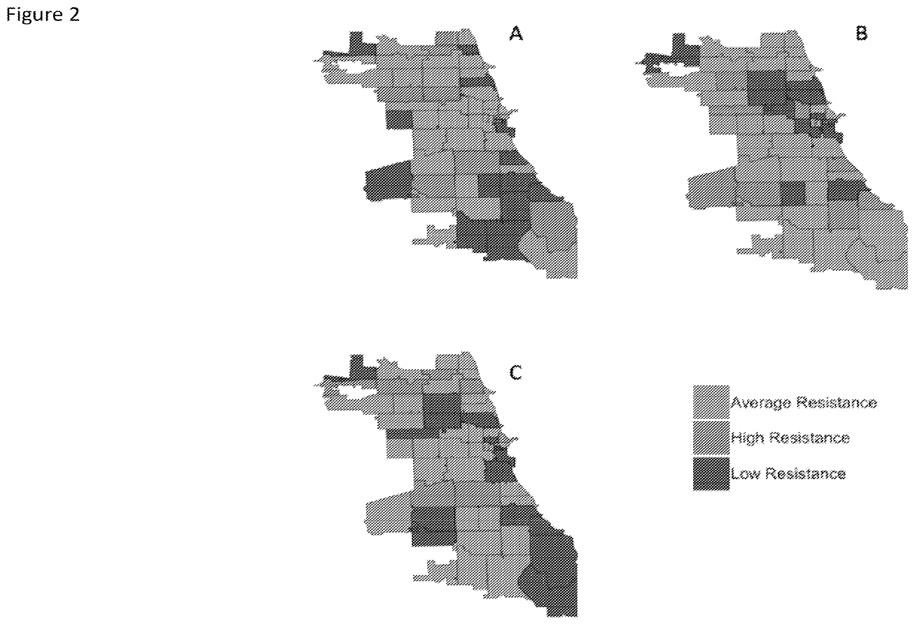

In the cohort, 4,849 patients with positive cultures lived in Chicago ZIP codes. Each ZIP code was given only one label of average, low or high resistance for each antimicrobial. A ZIP code was considered to be of average resistance for a certain antimicrobial if the proportion resistant for the whole cohort was contained within one standard error of the proportion resistant to the same antimicrobial in the ZIP code. If the proportion resistant in the ZIP code was below or above the average resistance for the whole cohort and did not contain the proportion resistant for the whole cohort within one standard error, the ZIP code was considered to be of low or high resistance, respectively. Of the 57 ZIP codes, 15 (26.3%) showed average resistance to all three drugs, four (7.0%) showed low resistance to all three drugs and none showed high resistance to all three drugs (FIG. 2).

3) Predictors of Antimicrobial Resistance

a) SXT

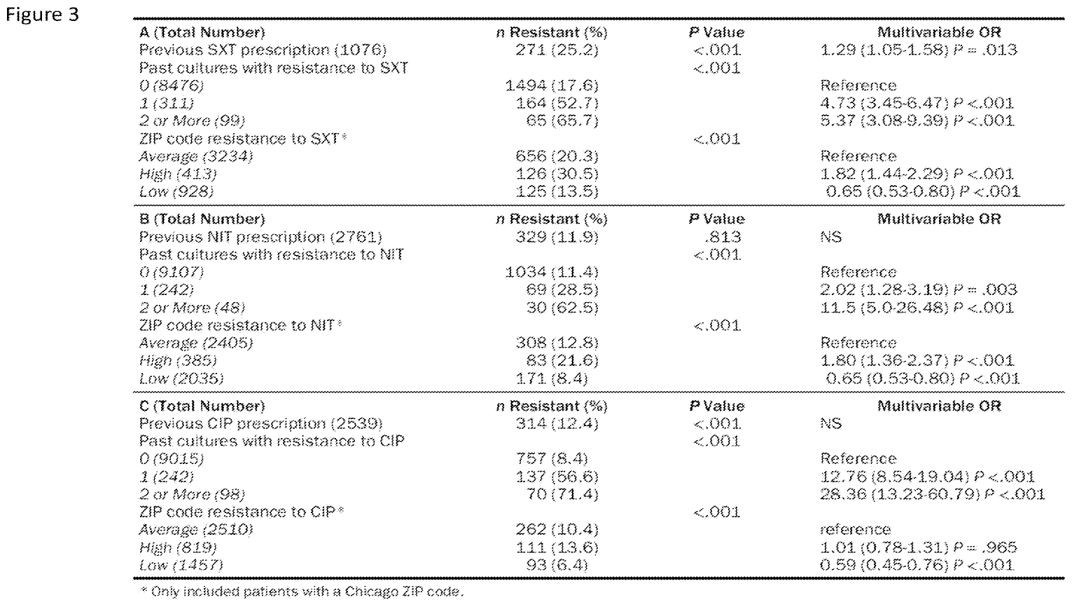

Age≥60 (OR 1.15 [95% CI 1.03-1.25] p=0.013), Asian race (OR 1.87 [95% CI 1.49-2.33] p<0.001) and a prior positive culture (OR 1.16 [95% CI 1.02-1.32] p=0.029) were associated with an increased likelihood of resistance to SXT on univariate analysis only. On multivariable analysis, a prescription of SXT within the past two years, having one or more previous cultures resistant to SXT and living in a ZIP code with a higher than average resistance prevalence to SXT, were all associated with an increased odds of resistance to SXT on the current culture and living in a ZIP code with a lower than average resistance prevalence to SXT was associated with a decreased odds of resistance to SXT on the current culture (FIG. 3A).

b) NIT

Age≥60 (OR 1.62 [95% CI 1.42-1.83] p<0.001) and Black race (OR 1.40 [95% CI 1.13-1.72] p=0.002) were associated with an increased likelihood of resistance to NIT on univariate analysis only. On multivariable analysis, having one or more previous cultures resistant to NIT and living in a ZIP code with a higher than average resistance prevalence to NIT were associated with an increased odds of resistance to NIT on the current urine culture and living in a ZIP code with a lower than average resistance prevalence to NIT was associated with a decreased odds of resistance to NIT on the current culture. A previous prescription of NIT did not increase the odds of resistance to NIT on the current culture. (FIG. 3B).

c) CIP

Age≥60 (OR 2.56 [95% CI 2.24-2.93] p<0.001), Asian race (OR 1.45 [95% CI 1.09-1.94] p=0.009), a prior positive culture (OR 1.33 [95% CI 1.12-1.59] p=0.001), a prescription of CIP within the past two years (OR 1.34 [95% CI 1.16-1.54] p<0.001) and living in a ZIP code with a higher than average resistance prevalence to CIP (OR 1.35 [1.06-1.70] p=0.014) were associated with an increased likelihood of resistance to CIP on univariate analysis only. On multivariable analysis, having one or more previous cultures resistant to CIP was associated with an increased odds of resistance to CIP on the current culture and living in a ZIP code with a lower than average resistance prevalence to CIP was associated with a decreased odds of resistance to CIP on the current culture (FIG. 3C).

4) 2011-2017 Cohort (Algorithm Development Population)

In examining patients from 2011-2017 with susceptibility data on SXT, NIT, CIP and a city of Chicago ZIP code, algorithms with NIT as the first option outperformed algorithms with SXT as the first option (i.e. NIT should be evaluated as an appropriate choice before SXT). An algorithm was created using previous resistance and past prescription data only for patients with prior microbiologic data, and for patients without prior microbiologic data an algorithm was created using only ZIP code data (FIG. 4). Prescription data on NIT was not used, as it was not a significant predictor of resistance on univariate or multivariable analysis. ZIP code data did not improve results or outperform the algorithm that used pervious resistance and past prescriptions for patients with prior microbiologic data.

5) 2018 Cohort (Testing Population)

Of the 2018 patients, 1,427 women had positive cultures, susceptibility data to SXT, NIT and CIP, a city of Chicago ZIP code and an antimicrobial prescribed during the encounter. Of these, 314 (22.0%) were resistant to SXT, 142 (10.0%) were resistant to NIT and 152 (10.7%) were resistant to CIP. The provider choices of empiric therapy against the index pathogen covered the pathogen empirically in 1,249 (87.5%) patients.

6) Algorithm Testing

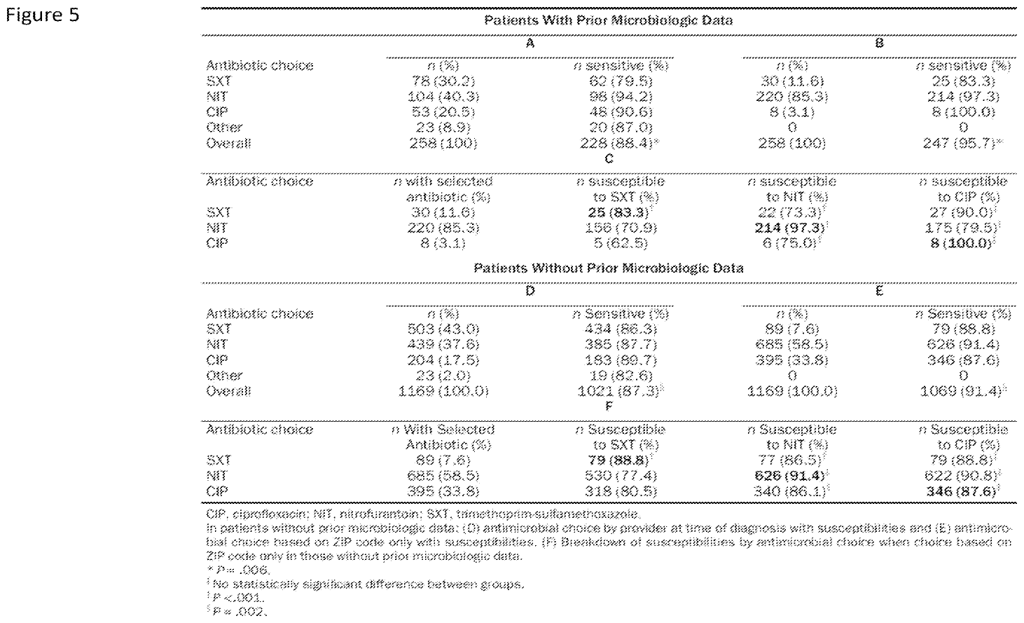

For the 258 patients for whom prior microbiologic data were available, the provider choices of empiric therapy against the index pathogen were accurate in 228 (88.4%) patients (FIG. 5A). When antimicrobial choice was made using the algorithm based on previous resistance and prior prescriptions (FIG. 4A), 247 (95.7%) patients were theoretically prescribed a drug to which their culture was susceptible, which was a statistically significant improvement from the actual antimicrobial prescribed (p=0.003) (FIG. 5B). It was also a statistically significant improvement from the theoretical scenario of all patients receiving SXT (72.1%, p<0.001), but not a significant improvement from all patients being prescribed NIT (93.8%, p=0.430). After applying this algorithm, the groups of patients created for each antimicrobial choice were evaluated, and among each group, the antimicrobial of choice had the highest proportion sensitive when compared to the other two antimicrobials or the difference was not statistically significant (FIG. 5C).

For the 1,169 patients for whom prior microbiologic data was not available, the provider choices of empiric therapy against the index pathogen were accurate in 1,021 (87.3%) patients (FIG. 5D). When antimicrobial choice was made using the algorithm based on ZIP code alone (FIG. 4B), 1,069 (91.4%) patients were theoretically prescribed a drug to which their culture was susceptible, which was a statistically significant improvement from the actual antimicrobial prescribed (p=0.002) (FIG. 5E). It was also a statistically significant improvement from the theoretical scenario of all patients being prescribed SXT (79.3%, p<0.001), but not a significant improvement from all patients being prescribed NIT (89.2%, p=0.080). After applying this algorithm, the groups of patients created for each antimicrobial choice were evaluated, and among each group the antimicrobial of choice either had the highest proportion sensitive when compared to the other two antimicrobials or the difference was not statistically significant (FIG. 5F).

6) All Patients

a. In combining data from the algorithms used for the 1,427 patients with and without prior microbiologic data, 1,316 (92.2%) patients were theoretically prescribed a drug to which their culture was susceptible, which was a statistically significant improvement from the actual antimicrobial prescribed (87.5%, p<0.001), if everyone was prescribed SXT (78.0%, p<0.001) and if everyone was prescribed NIT (90.0%, p=0.048).

D. Discussion

In this single medical system retrospective, cross-sectional study, we examined the value of a patient's history and demographic data in guiding empiric antimicrobial therapy for uncomplicated UTIs. We found patients' previous uropathogen resistance, antimicrobial prescriptions within two years and place of residence (ZIP code) to be highly predictive of antimicrobial resistance on subsequent cultures. In addition, sensitivity to NIT was high, suggesting that a clinical practice of only prescribing NIT would likely have favorable results. An algorithm using previous resistance, antimicrobial prescriptions within the past two years and ZIP code data was found to have a statistically significant improvement in selecting an appropriate antimicrobial for uncomplicated UTIs, compared to actual provider choice and theoretical guideline choice. These data suggest that existing data can be used to develop improved algorithms for empiric therapy for uncomplicated UTIs.

1) Correspondence of Age and Race

It has been shown that older patients tend to have a higher risk of resistance and some studies show racial groups may be predictors of antimicrobial resistance, although the results are inconsistent.^{19, 20} We found older age to be associated with an increased risk of antimicrobial resistance, as well as race, depending on the antimicrobial. Although these differences are statistically significant, they are small and the clinical utility is unclear.

2) Correspondence of Previous Resistance and Prior Prescriptions

Studies of patients with UTIs have shown that previous uropathogen resistance to an antimicrobial is associated with resistance in a subsequent UTI.⁸⁻¹¹ We showed that previous resistance to a particular antimicrobial is the strongest predictor of resistance to the same antimicrobial on a subsequent urine culture. This highlights the utility of considering uropathogen resistance in previous cultures in patients with recurrent UTIs.

Although there is little primary data, many studies and guidelines suggest that prior antimicrobial prescriptions are useful in guiding antimicrobial therapy in patients with UTIs.^{5, 6, 11} Our data reflects these trends: prescriptions within two years for SXT and CIP are associated with subsequent resistance to SXT and CIP, respectively, while a prior prescription for NIT is not associated with subsequent resistance. Our data support the concept that previous exposure to antimicrobials can help guide the selection of therapy in patients with uncomplicated UTIs.

3) Geographic Distribution of Antimicrobial Resistance

Previous studies, that have observed evidence of geographic clustering of resistance patterns of antimicrobial resistance among uropathogens, did show a slight correlation between areas with high levels of resistance to CIP and those with high levels of resistance to SXT, although these were based on larger geographic areas.¹² In this study, we showed that antimicrobial resistance by ZIP code is varied among different antimicrobials, and ZIP code is strongly associated with antimicrobial resistance on both univariate and multivariable analysis. These observations are consistent with the clonal theory in community acquired UTIs.²¹ Other factors such as socioeconomic status and antimicrobial utilization could also impact resistance by ZIP code. In using precise geographic areas to predict resistance, each antimicrobial will most likely need its own profile, as one distribution of resistance does not predict another.

4) Using Algorithms to Decide Therapy

In previous studies examining how an algorithm can be used to enhance antimicrobial selection among patients with UTIs, a key finding was that results improved significantly from guidelines if the patients had previous culture data and the choice was made using resistance patterns from those results.^{8, 10} Our data showed using previous resistance data combined with prior prescription data had a success rate of 95.7%. Although this was not a statistically significant improvement from the theoretical scenario of all patients receiving NIT, it was a significant improvement from the choices of the providers.

Although the gains are modest (approximately 7% for those with prior microbiologic data available), the implementation costs should be minimal. Also, we may be missing some of the benefit, as providers may already have considered past cultures when selecting treatments. We would expect a larger percentage of improvement in a cohort where no providers considered past cultures, compared to one that considered past cultures.

For patients who did not have previous culture data available, an algorithm using ZIP code data was able to achieve a success rate of 91.4%. This was an improvement from the real-world antimicrobial choices, but not a statistically significant improvement from the theoretical scenario of all patients receiving NIT (89.2%).

Interestingly, when the data from these two groups were combined, the success rate of 92.2% was a statistically significant improvement from both the theoretical scenarios (90.0% for NIT and 78.0% for SXT) and the real-world outcomes (87.5%). This highlights how collection of simple data points can have a strong effect in improving the delivery of care for patients with uncomplicated UTIs. It also highlights that in settings where this data may not be available, there is high utility in the use of NIT for all patients with uncomplicated UTIs.

5) Limitations

This study is limited by its retrospective design, since we were only able to incorporate patients with diagnosis codes of uncomplicated UTIs whose providers chose to obtain a urine culture. It is possible that patients with asymptomatic bacteriuria were included by providers who used the diagnosis codes incorrectly, but our chart review showed 96% of patients had documented symptoms. Also, this study does not incorporate patients' symptoms and follow-up, as many patients treated for a UTI with a particular antimicrobial who ultimately have resistance to that antimicrobial may still have symptomatic relief. Since many women are treated for symptoms of a UTI without a urine culture being obtained, it is difficult to confirm that we have evaluated the true prevalence of resistance in this community. Lastly, these findings will require confirmation at other sites.

6) Conclusions

Previous uropathogen resistance to an antimicrobial, prior antimicrobial prescriptions and ZIP codes are predictive of subsequent antimicrobial resistance. This information is available in EMRs. Algorithms utilizing these data can provide the best outcomes for empiric therapy for women with uncomplicated UTIs. This approach may result in increased confidence and accuracy for those who choose to treat immediately and antimicrobial stewardship (better coverage without using broader spectrum drugs), as well as patient satisfaction due to accurate treatment and reducing second visits due to ineffective initial therapy.

E. References for Example 2
1. Gupta, K., Trautner, B. W.: Diagnosis and management of recurrent urinary tract infections in non-pregnant women. BMJ, 346: f3140, 2013
2. Foxman, B.: The epidemiology of urinary tract infection. Nat Rev Urol, 7: 653-660, 2010
3. Foxman, B.: Urinary Tract Infection: Self-Reported Incidence and Associated Costs. Ann Epidemiol., 10: 509-515, 2000
4. Suskind, A. M., Saigal, C. S., Hanley, J. M. et al.: Incidence and Management of Uncomplicated Recurrent Urinary Tract Infections in a National Sample of Women in the United States. Urology, 90: 50-55, 2016
5. Gupta, K., Hooton, T. M., Naber, K. G. et al.: International clinical practice guidelines for the treatment of acute uncomplicated cystitis and pyelonephritis in women: A 2010 update by the Infectious Diseases Society of America and the European Society for Microbiology and Infectious Diseases. Clin Infect Dis, 52: e103-120, 2011
6. Hooton, T. M., Gupta, K.: Acute Simple Cystitis In Women. UpToDate, Waltham, Mass. (Accessed Jan. 31, 2019), 2018
7. Huttner, A., Verhaegh, E. M., Harbarth, S. et al.: Nitrofurantoin revisited: a systematic review and meta-analysis of controlled trials. J Antimicrob Chemother, 70: 2456-2464, 2015
8. Jackson, H. A., Cashy, J., Frieder, O. et al.: Data mining derived treatment algorithms from the electronic medical record improve theoretical empirical therapy for outpatient urinary tract infections. J Urol, 186: 2257-2262, 2011
9. MacFadden, D. R., Ridgway, J. P., Robicsek, A. et al.: Predictive utility of prior positive urine cultures. Clin Infect Dis, 59: 1265-1271, 2014
10. Linsenmeyer, K., Strymish, J., Gupta, K.: Two Simple Rules for Improving the Accuracy of Empiric Treatment of Multidrug-Resistant Urinary Tract Infections. Antimicrob Agents Chemother, 59: 7593-7596, 2015
11. Dickstein, Y., Geffen, Y., Andreassen, S. et al.: Predicting Antibiotic Resistance in Urinary Tract Infection Patients with Prior Urine Cultures. Antimicrob Agents Chemother, 60: 4717-4721, 2016
12. Galvin, S., Bergin, N., Hennessy, R. et al.: Exploratory Spatial Mapping of the Occurrence of Antimicrobial Resistance in E. coli in the Community. Antibiotics, 2: 328-338,
13. Sannes, M. R., Kuskowski, M. A., Johnson, J. R.: Geographical distribution of antimicrobial resistance among Escherichia coli causing acute uncomplicated pyelonephritis in the United States. FEMS Immunol Med Microbiol, 42: 213-218, 2004
14. McCormick, A. W., Whitney, C. G., Farley, M. M. et al.: Geographic diversity and temporal trends of antimicrobial resistance in Streptococcus pneumoniae in the United States. Nat Med, 9: 424-430, 2003
15. Cohen, J. E., Yura, E. M., Chen, L. et al.: Predictive Utility of Prior Negative Urine Cultures in Women with Suspected Recurrent Uncomplicated Urinary Tract Infections. J Urol: 101097JU0000000000000325, 2019
16. Gupta, K., Grigoryan, L., Trautner, B.: Urinary Tract Infection. Ann Intern Med, 167: ITC49-ITC64, 2017
17. Hooton, T. M., Roberts, P. L., Cox, M. E. et al.: Voided midstream urine culture and acute cystitis in premenopausal women. N Engl J Med, 369: 1883-1891, 2013
18. Giesen, L. G., Cousins, G., Dimitrov, B. D. et al.: Predicting acute uncomplicated urinary tract infection in women: a systematic review of the diagnostic accuracy of symptoms and signs. BMC Fam Pract, 11: 78, 2010
19. Rattanaumpawan, P., Nachamkin, I., Bilker, W. B. et al.: Risk factors for ambulatory urinary tract infections caused by high-MIC fluoroquinolone-susceptible Escherichia coli in women: results from a large case-control study. J Antimicrob Chemother, 70: 1547-1551, 2015
20. Delisle, G., Quach, C., Domingo, M. C. et al.: Escherichia coli antimicrobial susceptibility profile and cumulative antibiogram to guide empirical treatment of uncomplicated urinary tract infections in women in the province of Quebec, 2010-15. J Antimicrob Chemother, 71: 3562-3567, 2016
21. Smith, S. P., Manges, A. R., Riley, L. W.: Temporal changes in the prevalence of community-acquired antimicrobial-resistant urinary tract infection affected by Escherichia coli clonal group composition. Clin Infect Dis, 46: 689-695, 2008

### Example 3: Algorithms to Enhance Empiric Antibiotic Choice for Complicated Urinary Tract Infection Reflects Importance of Status of the Urinary Tract and Location of Acquisition

Purpose: To determine predictive factors for antimicrobial resistance patterns and to develop an antimicrobial treatment algorithm for outpatients presenting with a complicated UTI.

Materials and Methods: We performed a retrospective, single-center, cross-sectional study of 2,286 outpatients with a diagnosed complicated UTI from 2012-2018. For patients with confirmed UTI (≥10³ CFU/mL) with antimicrobial sensitivities, univariate analyses and multivariable regression models were used to determine odds ratios for predicting resistance to SXT, NIT, CIP, CF-1, and CF-3 for the 2012-2016 data. Antimicrobial choice algorithms were created using 2012-2016 results and tested on 2017-2018 data.

Results: For outpatients presenting with complicated UTI, overall prevalence of resistance for SXT, CIP, NIT, CF-1 and CF-3 was 25.5%, 19.4%, 18.9%, 15.2% and 7.0%, respectively. Consistent predictive factors influencing resistance to all five antimicrobials were ZIP code, status of host urinary tract (complicated vs. uncomplicated), and prior resistance to the antimicrobial. Resulting treatment algorithm for complicated UTIs (whether or not prior microbiologic data was available) outperformed real-life provider choice and our previously published algorithm for uncomplicated UTIs.

Conclusions: Treatment algorithms for UTIs are dependent on location of infection acquisition (ZIP code), status of the host urinary tract (complicated or uncomplicated), and prior urine culture resistance data. When using our complicated UTI treatment algorithm regardless of uropathogen, our results outperformed real-life scenario provider choice and our prior published algorithm for uncomplicated UTI, which can help guide empiric antibiotic choice.

Introduction

Urinary tract infection (UTI) is the most common acquired outpatient infection in the United States and is highly prevalent worldwide^{1,2}. Complicated UTIs (i.e. UTIs in patients with underlying urologic abnormalities, recent instrumentation (healthcare-associated UTI), immunocompromising conditions or poorly controlled diabetes mellitus) while not as prevalent as uncomplicated UTIs impose unique challenges to providers³⁻⁶. Antimicrobial selection for complicated UTIs is challenging given higher prevalence of resistant organisms, prior antibiotic use, healthcare-related exposures, and increased incidence of concomitant fever or sepsis making timely, empiric antibiotic selection critical⁷⁻¹¹. Furthermore, guidelines for antibiotic selection for outpatient complicated UTIs are limited and often confusing for providers given the increasing resistance to antimicrobials locally and globally, the heterogeneity of in vitro susceptibility patterns by geographic region, and the available treatment nomograms for providers often represent an admixture of antimicrobial data, in which resistance patterns are skewed by hospital acquired infections^{5,12-16.} These factors make it difficult for providers to make an empiric antibiotic choice, specifically for community acquired, complicated UTIs.

For uncomplicated UTIs, our research group built upon historic treatment guidelines and concepts established in prior studies and demonstrated that antimicrobial resistance varies by ZIP code (presumed location of infection acquisition) and this data, along with prior culture history, can be used to guide empiric antibiotic therapy in a way that outperforms real-world provider choices^{10,17-23}. Our goal herein was to devise a similar treatment algorithm to guide empiric antibiotic choice for outpatient complicated UTIs. We first hypothesized that the treatment algorithm for complicated UTI would differ from uncomplicated UTI to reflect importance of status of the urinary tract. Secondly, we hypothesized that algorithms using location of UTI acquisition (ZIP code), prior culture resistance and antimicrobial prescription history could guide empiric antimicrobial therapy for treatment of complicated UTIs and outperform real world outcomes.

Materials and Methods

This study was approved and performed in accordance with the Northwestern Medicine Institutional Review Board (STU00207763). This is a retrospective cross-sectional study of patients aged 18 or older that submitted urine cultures as outpatients from 2012 to 2018 at any Northwestern Medicine site, which encompasses the greater Chicago area. Data were obtained from the Northwestern Medicine Enterprise Data Warehouse, which is a data integration warehouse designed for research and clinical operations. Patients were included for analysis only if the encounter at which the urine culture was collected had an ICD 9 (599.0 & 595) or 10 (N39.0 & N30) diagnosis code for UTI. Patients and cultures were included only if they were linked to any ICD 9 or 10 diagnosis code in the previous two years that would have been associated with a complicated UTI⁶. Patients were included exactly once based on their most recent culture, and all prior culture data were used as covariates. Patients were excluded if they had a hospital admission or surgical encounter in the previous 30 days. Cultures were included in analysis if positive (identified uropathogen≥10³ colony forming units per milliliter (CFU/mL)) and antimicrobial susceptibilities were reported for the dominant species. All specimen collection and testing were done uniformly in accordance with Northwestern Medicine Guidelines. Isolation, colony count and identification testing were performed using Vitek 2 cards (bioMérieux).

For the 2012 to 2016 patients, outcomes of interest were uropathogen resistance (defined as resistant or intermediate susceptibility) to trimethoprim-sulfamethoxazole (SXT), ciprofloxacin (CIP), nitrofurantoin (NIT), first-generation cephalosporin (CF-1) and third-generation cephalosporin (CF-3). The resistance prevalence was also stratified by Chicago ZIP code for each antimicrobial. For each antimicrobial, a ZIP code was considered low or high resistance if the prevalence of resistance in that ZIP code was below or above the average, respectively, for the whole city and did not contain the average within one standard error.

Evaluating Outpatient Complicated UTI Population

Covariates included patient demographics (age, race and ZIP code of residence), any antimicrobial prescriptions within the past two years, diagnosis codes associated with current and past encounters, past urine culture results and department to which patient presented. Risk factors were compared between patients who had resistant and sensitive cultures using two-sample t-tests (continuous variables) and chi-squared tests (categorical variables). Multivariate logistic regression models were used to investigate the association between risk factors and resistant cultures. Odds ratios (OR) were derived from these models to determine the probability of having a resistant culture with those factors. Algorithms were then created using the risk factors found to be most predictive of a resistant culture in the analysis described above.

Testing Population

The algorithms developed using the above-described cohort were then tested on the 2017-2018 cohort. The overall success of these algorithms (proportion with a chosen antimicrobial that had a culture susceptible to that drug) was determined and compared to provider-chosen therapy and guideline chosen therapy. Also, the predictive value of time between cultures and uropathogen concurrence between cultures was examined in this cohort^{10,17}. All analyses were done using R (version 3.5.2).

Comparing Resistance in Different Populations

The prevalence of resistance for patients with complicated UTIs was compared to data from our previous publication for uncomplicated UTIs and available hospital data¹⁷. The prevalence of resistance by ZIP code for the complicated UTI population was then compared between each antibiotic and to the distribution of prevalence by ZIP code for the uncomplicated population for the previous study, for CIP, SXT and NIT (the three antimicrobials evaluated in the previous study)¹⁷.

Results

Increased Antimicrobial Resistance Among Complicated UTI Population Compared to Uncomplicated UTI

Over the study period, 2,286 patients were diagnosed with complicated UTIs as outpatients and had a positive urine culture (≥10³ CFU/mL) with documented antimicrobial susceptibilities. Mean patient age was 60.2 years. 712 (31.1%) had a prior positive culture and 1,571 (68.8%) were white. The most common departments at which the cultures were obtained were the Emergency Department or Immediate Care (37.2%), Internal Medicine (16.3%) and Urology (9.8%). The most common uropathogens were Escherichia coli (61.4%), Enterococcus spp. (11.9%) and Klebsiella pneumoniae (9.6%). Of the 2,286 patients, 41 (1.8%) had a fever (>100.4° F., maximum was 101.8° F.), which were included in the analysis.

Of the 2,286 cultures, 1,892 (82.8%), 2,195 (96.0%), 2,246 (98.3%), 1,901 (83.2%) and 1,906 (83.4%) had susceptibilities reported to SXT, CIP, NIT, CF1 and CF3, respectively. Overall prevalence of resistance for SXT, CIP, NIT, CF1 and CF3 was 25.5%, 19.4%, 18.9%, 15.2% and 7.0%, respectively. Resistance was stable throughout the study period for these antimicrobials.

Compared to complicated UTIs, antimicrobial resistance for uncomplicated UTIs (previously published) was significantly lower for all drugs assessed in both studies: SXT (19.4%), CIP (10.3%), and NIT (12.1%) (p<0.001, p<0.001 and p<0.001, respectively)¹⁷.

Antimicrobial Resistance Varies by ZIP Code (Location of Infection Acquisition)

In the cohort, 1,300 patients with positive cultures (56.9%) lived within a Chicago ZIP code. Each ZIP code was given a label of low, average, or high resistance for each antimicrobial. Average resistance for an antimicrobial within a ZIP code was defined as within one standard error of proportion resistant to the same antimicrobial in the whole cohort. If the proportion resistant within the ZIP code was below or above the average resistance (beyond one standard error) for the whole cohort, the ZIP code was considered to be of low or high resistance, respectively¹⁷.

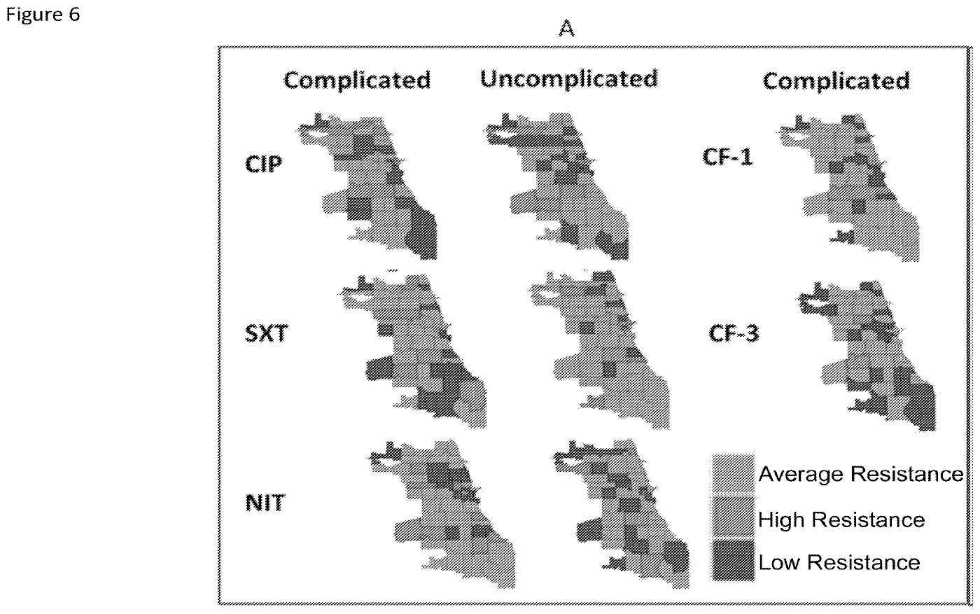

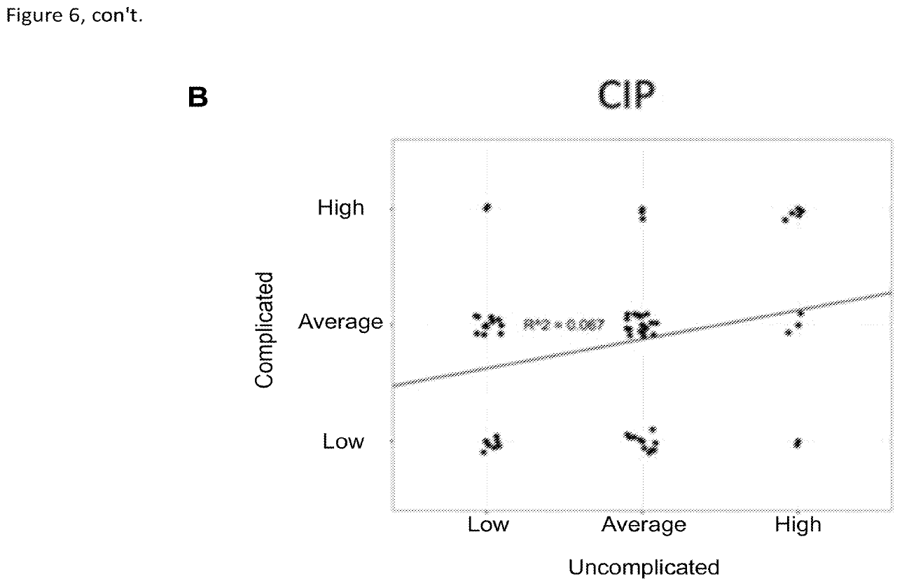

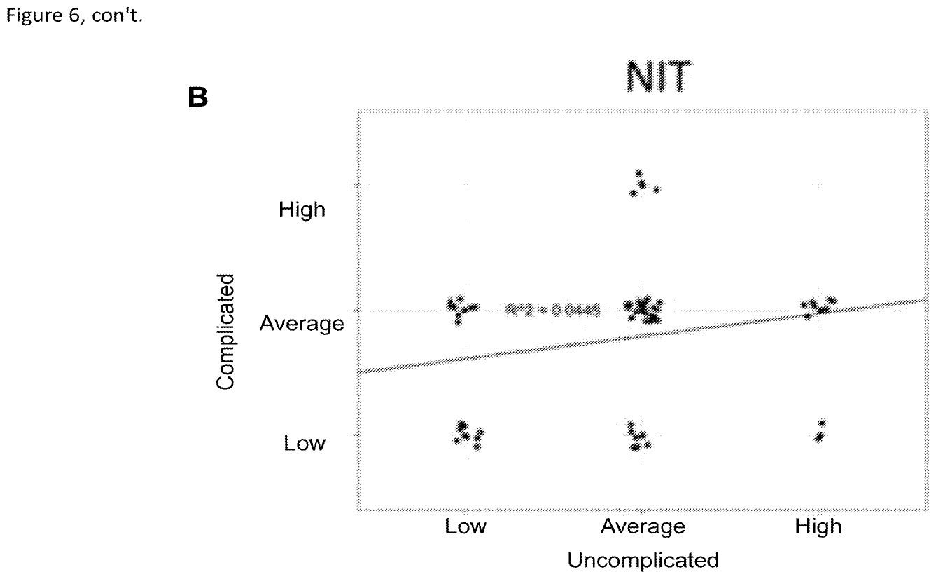

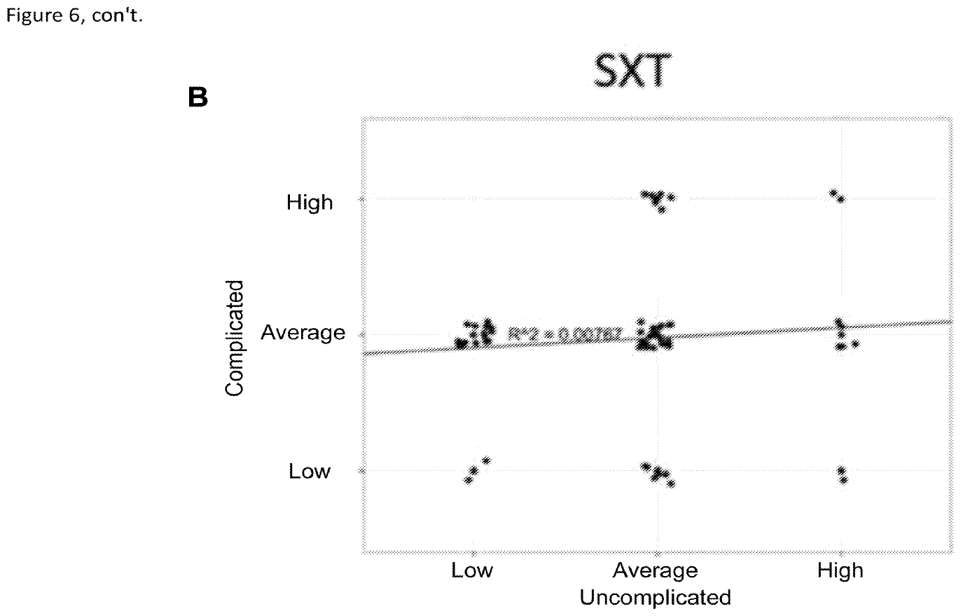

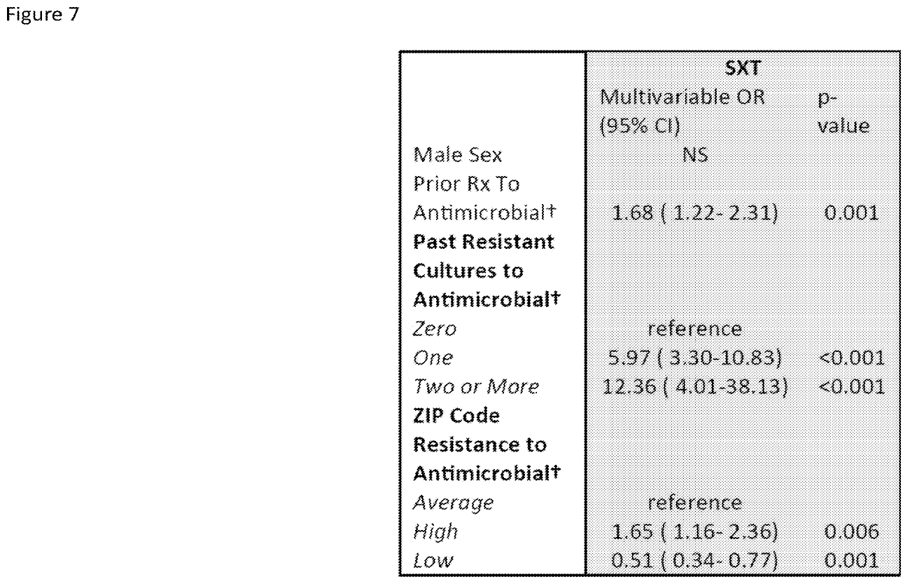

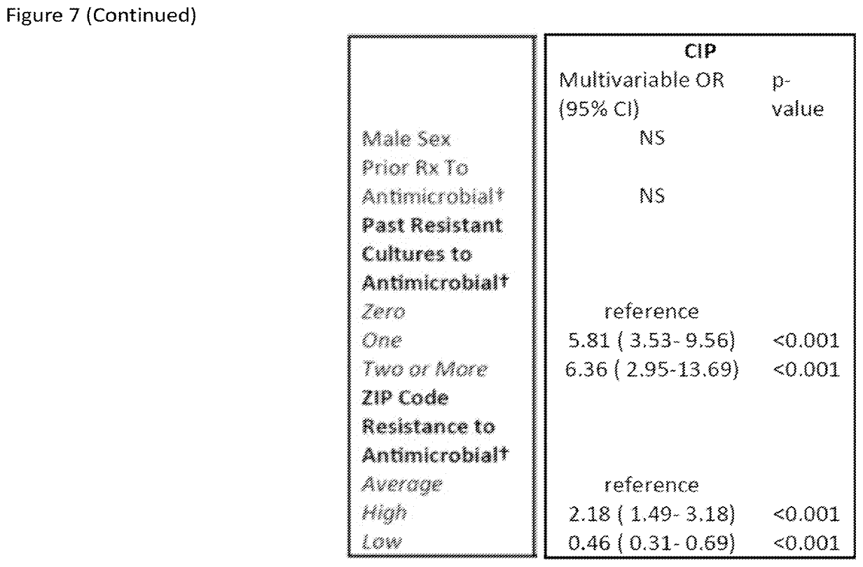

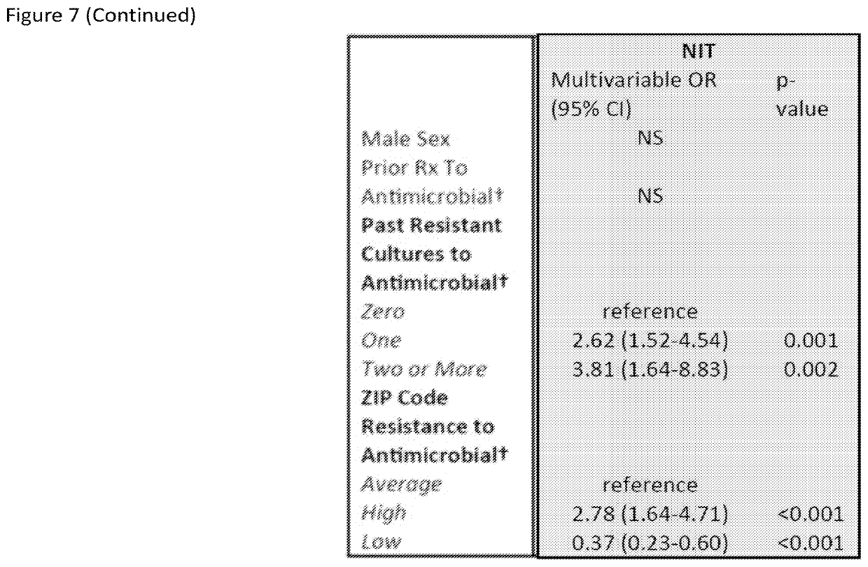

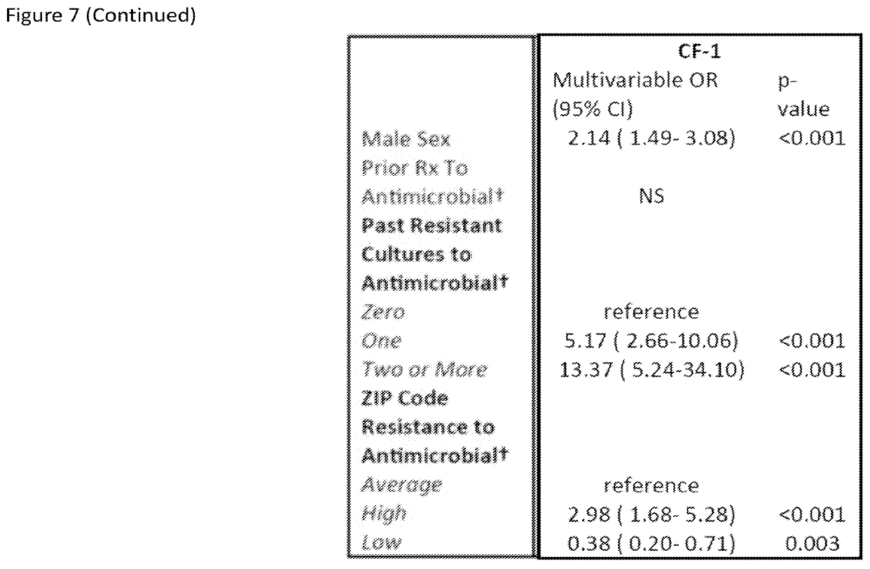

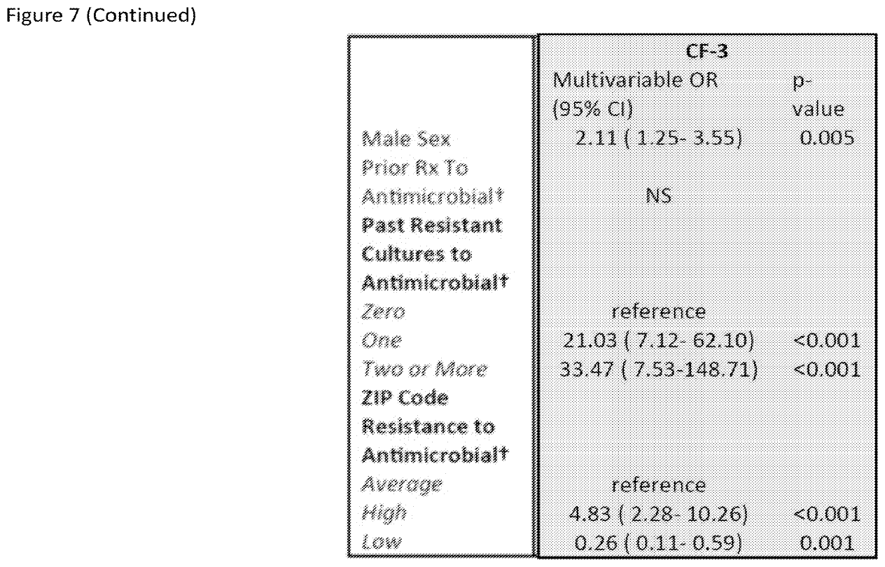

The resistance prevalence for each antimicrobial varied greatly between ZIP codes. Of the 57 ZIP codes in the city of Chicago for which we had patient data, 1 (1.8%) had resistance prevalence below the average for all five antimicrobials and no ZIP codes were above average for all five antimicrobials. Four (7.0%) had low resistance for four of the five antimicrobials and 1 (1.8%) had high resistance for four of the five antimicrobials (FIG. 6A). On univariate and multivariable analysis, ZIP code was a significant predictor of resistance to each of the five antimicrobials (FIG. 7).

Antimicrobial Resistance Varies by Status of the Urinary Tract (Complicated Vs. Uncomplicated)

The status of the urinary tract (complicated vs. uncomplicated) influenced antimicrobial resistance. Compared to complicated UTI, overall antimicrobial resistance (SXT, NIT, and CIP) was significantly lower for uncomplicated UTI (data from our prior study) (p<0.001 for each drug) (FIG. 6A)¹⁷. When comparing antimicrobial resistance by ZIP code for uncomplicated vs. complicated UTI, there was no significant correlation between the two populations for SXT, NIT, or CIP (FIG. 6B). There was also no significant correlation between different antimicrobials within the same populations (data not shown).

According to Northwestern Hospital uropathogen resistance data for 2018, which includes both inpatient and outpatient cultures, the overall prevalence of resistance to CIP was 25%. Our data for CIP resistance for community acquired UTIs, both uncomplicated and complicated, for the same year was 11.2% (CIP resistance: 10.7%-uncomplicated; 16.2%-complicated).

Other Predictors of Antimicrobial Resistance

Race: On univariate analysis only, Asian race was a significant predictor of resistance for SXT, compared to white race (OR 2.22 [95% CI: 1.26-3.92], p=0.006) while black race was a significant predictor of resistance for NIT, compared to white race (OR 1.37 [95% CI: 1.01-1.87], p=0.045) (data not shown).

Prior positive urine culture: On univariate analysis only, a prior positive urine culture was a significant predictor of resistance for all of the selected antimicrobials (SXT OR 1.45 [95% CI: 1.15-1.68], p=0.002; CF1 OR 1.28 [95% CI: 1.06-1.43], p=0.040; CF3 OR 1.34 [95% CI: 1.08-1.54], p=0.043) other than NIT and CIP (data not shown).

Age: Age 60 or older was a significant predictor of resistance for CIP (OR 1.73 [95% CI: 1.39-2.16], p<0.001) and NIT (OR 1.26 [95% CI: 1.02-1.57], p=0.033) (data not shown)

Male sex: On both univariate and multivariable analysis, male sex was only a predictor of antimicrobial resistance to CF-1 and CF-3 compared to females within the same ZIP code (Table 1).

Prior prescription for an antimicrobial: On both univariate and multivariable analysis, past prescription for SXT alone was associated with increased current resistance to SXT (Table 1).

Past resistance to the antimicrobial: On both univariate and multivariable, past resistance to the antimicrobial of interest was a significant predictor of current resistance to that antimicrobial for all five antimicrobials. Compared to zero prior cultures as a reference range, increasing number of prior resistant cultures was significantly associated with current resistance regardless of uropathogen found in past or current urine culture (FIG. 7).

The time interval between the index culture and the culture for which prior microbiologic data were available was not associated with differences in the likelihood of resistance to any of the five antimicrobials on the current culture.

Algorithm Generation for Outpatient Complicated UTI

2012-2016 Cohort (Algorithm Development Population)

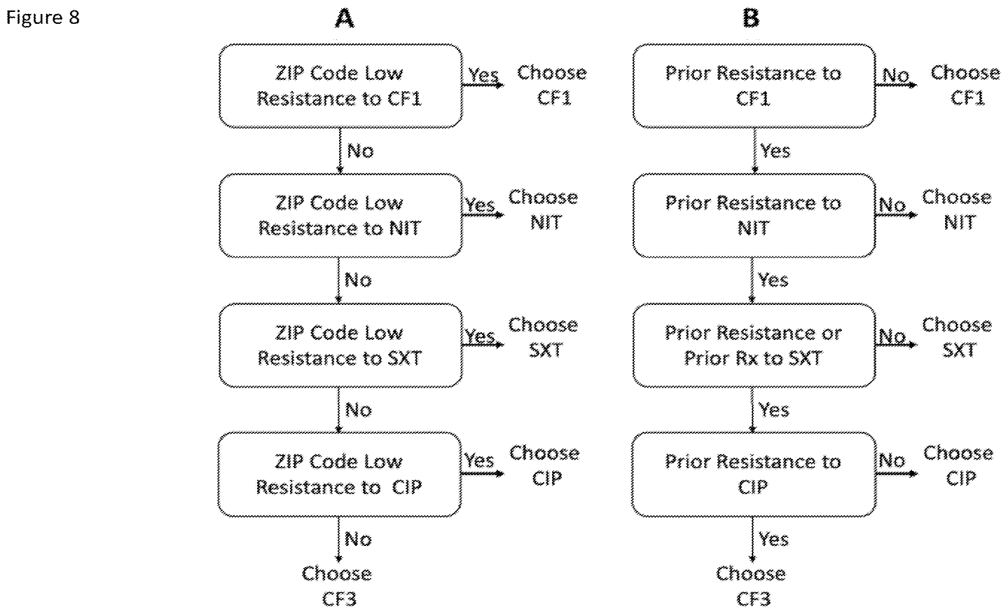

In examining outpatients presenting with complicated UTI only from 2012-2016 with susceptibility data for SXT, NIT, CIP, CF-1 and CF-3 and a city of Chicago ZIP code, algorithms with CF-1 as the first option outperformed algorithms with SXT, CIP or NIT as the first option (i.e. CF-1 should be considered for empiric antibiotic therapy in complicated UTI patients presenting without prior culture data before other antimicrobials). An algorithm was created using only ZIP code data for outpatients without prior microbiologic data to guide empiric antibiotic choice (FIG. 8A). A separate algorithm was generated for outpatients presenting with prior microbiologic data based on previous urine culture resistance patterns and past antibiotic prescription data only (FIG. 8B). Only prescription data on SXT was used in algorithm generation, as it was the only antimicrobial for which a past prescription was a significant predictor of resistance (FIG. 7).

2017-2018 Cohort (Testing Population)

Among outpatients presenting with complicated UTI from 2017-2018, 646 patients had positive urine cultures, susceptibility data to SXT, NIT, CIP, CF-1 and CF-3, a city of Chicago ZIP code and an antimicrobial prescribed during the encounter. Of these, 167 (25.9%) were resistant to SXT, 117 (18.1%) resistant to NIT, 119 (18.4%) resistant to CIP, 103 (15.9%) resistant to CF-1 and 54 (8.4%) resistant to CF-3. None of the patients were febrile at the time of culture. Overall, the provider choice of empiric antibiotic therapy against the index uropathogen was correct in 520 (80.5%) of patient encounters.

Algorithm Testing

Outpatients without Prior Microbiologic Data

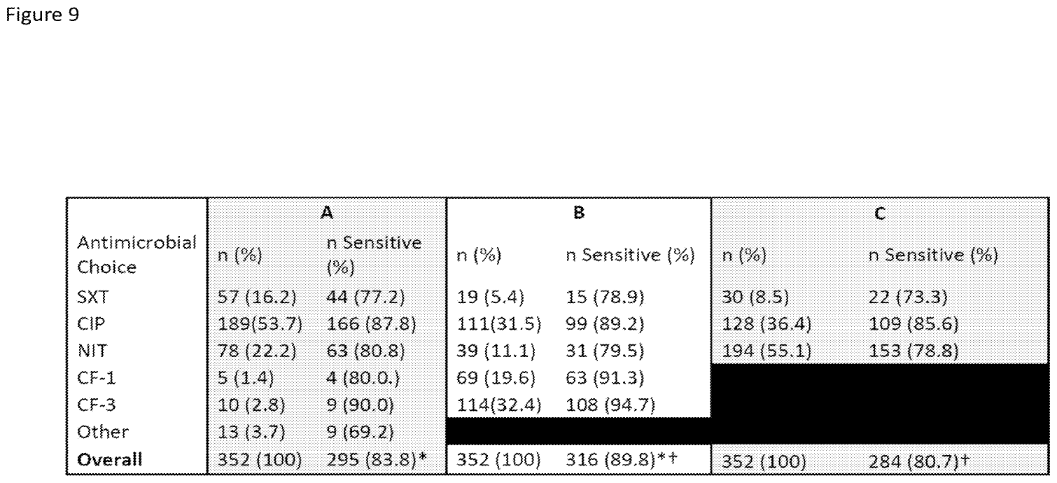

There were 352 patients presenting with an outpatient complicated UTI without prior microbiologic data. Without using the algorithm, the provider choice of empiric antibiotic therapy against the index uropathogen was accurate in 295/352 (83.8%) of patient encounters (FIG. 9A). If our treatment algorithm was used based on patient ZIP code alone (FIG. 8A), 316/352 (89.8%) patients would have been prescribed a susceptible antibiotic, which is a statistically significant improvement (p=0.026, FIG. 9B). If our previously published treatment algorithm for uncomplicated UTI had been used instead, 284/352 (80.7%) patients would have received a susceptible antibiotic, which is a statistically significant deterioration (p=0.002, FIG. 9C)¹⁷.

Outpatients with Prior Microbiologic Data

There were 294 patients presenting with a complicated UTI with prior microbiologic data available. Without using the algorithm, the provider choice of empiric antibiotic therapy against the index uropathogen was accurate in 225/294 (76.5%) of patient encounters. If our treatment algorithm was used based on previous resistance and prior prescriptions (FIG. 8B), 257/294 (87.4%) patients would have been prescribed a susceptible antibiotic, which is a statistically significant improvement (p<0.001). If our algorithm for complicated UTI based on ZIP code alone (FIG. 8A) had been used instead, 233/294 (79.3%) patients would have received a susceptible antibiotic, which is a statistically significant deterioration from the algorithm based on previous resistance and prior prescriptions (p=0.012) and not a statistically significant difference from the actual antimicrobial prescribed (p=0.487).

In combining data from the algorithms used for the 646 patients with complicated UTIs both with and without prior microbiologic data, 573/646 (88.7%) patients were theoretically prescribed a drug to which their culture was susceptible, which was a statistically significant improvement from the actual antimicrobial prescribed (80.5%, p<0.001).

### DISCUSSION

Choosing empiric antibiotic therapy for outpatients presenting with both complicated or uncomplicated UTIs can be challenging, but complicated UTIs can be particularly complex given higher prevalence of resistant organisms, heterogeneity of uropathogen susceptibility patterns by geographic region, and convoluted treatment nomograms that often represent an admixture of microbiologic data from inpatients and outpatients⁵⁻¹⁶. Our objectives were (1) to determine predictive factors of antimicrobial resistance patterns for patients with a complicated UTI and (2) to develop a treatment algorithm for outpatients presenting with a complicated UTI that could outperform current provider choice for empiric antibiotic therapy.

The only consistent predictive factors of resistance among all five antimicrobials (SXT, CIP, NIT, CF-1, CF-3) for patients with a UTI were prior resistance to the antimicrobial, ZIP code, and status of the host urinary tract (complicated vs. uncomplicated). Other factors such as race, gender, prior positive urine culture, prior prescription for a given antimicrobial, or time between urine cultures varied in their importance of predicting antimicrobial resistance and were not effective adjuncts in algorithm development.

For both uncomplicated (previously published) and complicated UTIs, ZIP code was an important predictor of antimicrobial resistance regardless of uropathogen; but, the resistance patterns were very different when comparing uncomplicated to complicated infections¹⁷. While we traditionally predict UTI resistance patterns by the location of infection acquisition (e.g. nosocomial, ICU, nursing home, community), we found different resistance patterns among each ZIP code (FIG. 6A). While location of acquisition is important, status of the urinary tract (uncomplicated vs. complicated) was similarly crucial in determining resistance patterns in the population (FIG. 6). Taken together, location of acquisition (ZIP code) and status of the host urinary tract were critical for accurate empiric antibiotic choice. FIG. 8A demonstrates the generated treatment algorithm for outpatients presenting with a complicated UTI without prior microbiologic data based on ZIP code alone. Our algorithm outperformed actual provider choice in the clinical scenario and if the provider were to use our previously published uncomplicated UTI algorithm¹⁷. As we expected from previous reports and clinical experience, prior urine culture resistance data can aid prescribers in choosing empiric therapy and so influenced our treatment algorithm as well (FIG. 8B)^{6, 10}.

Our study is limited by its retrospective design and being from a single center our algorithms are reflective of resistance patterns in the Chicago-land area. Our algorithm also presumes that outpatients acquired their infection within their home ZIP code. While this is an assumption, our algorithm still outperformed real-time provider choice and was correct in 89.8% of complicated UTI patients without prior microbiologic data and 87.4% with prior microbiologic data. Furthermore, just as the uncomplicated UTI algorithm did not accurately predict resistance patterns for complicated UTIs, our complicated UTI algorithm reflects a limited population: outpatients with complicated UTIs who are generally afebrile, non-septic. We also believe it is important to update/retest the algorithms periodically (e.g. yearly) to accommodate for changes in resistance patterns. Lastly, our algorithm only takes into account oral options for antibiotics. Although these factors limit wide-spread applicability, strengths of our paper include (1) algorithms for antimicrobial resistance are applicable regardless of prior and current uropathogen identified; (2) it provides Chicago-land prescribers with more algorithm-driven data to accurately choose empiric antibiotic therapy thus reducing guess-work especially for first-time complicated UTI patients; and (3) it clarifies that a one-drug-fits-all approach, even for a first time diagnosis, is not an effective strategy for the treatment of UTIs—but rather location of acquisition, status of the host urinary tract, and prior resistance data are critical for accurate antimicrobial stewardship.

Conclusion

Treatment algorithms for UTIs are dependent on location of infection acquisition (ZIP code), status of the host urinary tract (complicated or uncomplicated), and prior urine culture resistance data. When using our complicated UTI treatment algorithm, our results outperformed real-life scenario provider choice and our prior published algorithm for uncomplicated UTI. Our algorithm can be applied to complicated UTI patients, with or without prior urine culture data, in the Chicago-land area to help guide empiric antibiotic choice.

References for Example 3
1. Medina M, Castillo-Pino E. An introduction to the epidemiology and burden of urinary tract infections. Therapeutic Advances in Urology. 2019; 11:1756287219832172. doi:10.1177/1756287219832172
2. Vallejo-Torres L, Pujol M, Shaw E, et al. Cost of hospitalised patients due to complicated urinary tract infections: a retrospective observational study in countries with high prevalence of multidrug-resistant Gram-negative bacteria: the COMBACTE-MAGNET, RESCUING study. BMJ Open. 2018; 8(4): e020251. doi:10.1136/bmjopen-2017-020251
3. Nicolle L E, Gupta K, Bradley S F, et al. Clinical Practice Guideline for the Management of Asymptomatic Bacteriuria: 2019 Update by the Infectious Diseases Society of America. Clin Infect Dis. 2019; 68(10): e83-e110. doi:10.1093/cid/ciy1121
4. Foxman B. The epidemiology of urinary tract infection. Nat Rev Urol. 2010; 7(12):653-660. doi:10.1038/nruro1.2010.190
5. Abbo L M, Hooton T M. Antimicrobial Stewardship and Urinary Tract Infections. Antibiotics (Basel). 2014; 3(2):174-192. doi:10.3390/antibiotics3020174
6. Gupta K, Grigoryan L, Trautner B. Urinary Tract Infection. Ann Intern Med. 2017; 167(7): ITC49-ITC64. doi:10.7326/AITC201710030
7. Khawcharoenporn T, Vasoo S, Singh K. Urinary Tract Infections due to Multidrug-Resistant Enterobacteriaceae: Prevalence and Risk Factors in a Chicago Emergency Department. Emerg Med Int. 2013; 2013:258517. doi:10.1155/2013/258517
8. Killgore K M, March K L, Guglielmo B J. Risk factors for community-acquired ciprofloxacin-resistant Escherichia coli urinary tract infection. Ann Pharmacother. 2004; 38(7-8):1148-1152. doi:10.1345/aph.1D622
9. Walker E, Lyman A, Gupta K, Mahoney M V, Snyder G M, Hirsch E B. Clinical Management of an Increasing Threat: Outpatient Urinary Tract Infections Due to Multidrug-Resistant Uropathogens. Clin Infect Dis. 2016; 63(7):960-965. doi:10.1093/cid/ciw396
10. Linsenmeyer K, Strymish J, Gupta K. Two Simple Rules for Improving the Accuracy of Empiric Treatment of Multidrug-Resistant Urinary Tract Infections. Antimicrob Agents Chemother. 2015; 59(12):7593-7596. doi:10.1128/AAC.01638-15
11. Smithson A, Chico C, Ramos J, et al. Prevalence and risk factors for quinolone resistance among Escherichia coli strains isolated from males with community febrile urinary tract infection. Eur J Clin Microbiol Infect Dis. 2012; 31(4): 423-430. doi:10.1007/s10096-011-1322-y
12. Drekonja D M, Rector T S, Cutting A, Johnson J R. Urinary tract infection in male veterans: treatment patterns and outcomes. JAMA Intern Med. 2013; 173(1):62-68. doi:10.1001/2013.jamainternmed.829
13. Lob S H, Nicolle L E, Hoban D J, Kazmierczak K M, Badal R E, Sahm D F. Susceptibility patterns and ESBL rates of Escherichia coli from urinary tract infections in Canada and the United States, SMART 2010-2014. Diagn Microbiol Infect Dis. 2016; 85(4):459-465. doi:10.1016/j.diagmicrobio.2016.04.022
14. Hooton T M, Bradley S F, Cardenas D D, et al. Diagnosis, prevention, and treatment of catheter-associated urinary tract infection in adults: 2009 International Clinical Practice Guidelines from the Infectious Diseases Society of America. Clin Infect Dis. 2010; 50(5):625-663. doi:10.1086/650482
15. Tandogdu Z, Bartoletti R, Cai T, et al. Antimicrobial resistance in urosepsis: outcomes from the multinational, multicenter global prevalence of infections in urology (GPIU) study 2003-2013. World J Urol. 2016; 34(8):1193-1200. doi:10.1007/s00345-015-1722-1
16. Sanchez G V, Babiker A, Master R N, Luu T, Mathur A, Bordon J. Antibiotic Resistance among Urinary Isolates from Female Outpatients in the United States in 2003 and 2012. Antimicrob Agents Chemother. 2016; 60(5):2680-2683. doi:10.1128/AAC.02897-15
17. Cohen J E, Chen L, Schaeffer A J. Algorithms Using Previous Resistance, Prior Antimicrobial Prescriptions, and Patient Place of Residence Enhance Empirical Therapy for Women With Uncomplicated Urinary Tract Infections. Urology. 2020; 137:72-78. doi:10.1016/j.urology.2019.11.009
18. Galvin S, Bergin N, Hennessy R, et al. Exploratory Spatial Mapping of the Occurrence of Antimicrobial Resistance in E. coli in the Community. Antibiotics (Basel). 2013; 2(3):328-338. doi:10.3390/antibiotics2030328
19. Sannes M R, Kuskowski M A, Johnson J R. Geographical distribution of antimicrobial resistance among Escherichia coli causing acute uncomplicated pyelonephritis in the United States. FEMS Immunol Med Microbiol. 2004; 42(2):213-218. doi:10.1016/j.femsim.2004.05.004
20. McCormick A W, Whitney C G, Farley M M, et al. Geographic diversity and temporal trends of antimicrobial resistance in Streptococcus pneumoniae in the United States. Nat Med. 2003; 9(4):424-430. doi:10.1038/nm839
21. Jackson H A, Cashy J, Frieder O, Schaeffer A J. Data mining derived treatment algorithms from the electronic medical record improve theoretical empirical therapy for outpatient urinary tract infections. J Urol. 2011; 186(6):2257-2262. doi:10.1016/j.juro.2011.07.092
22. MacFadden D R, Ridgway J P, Robicsek A, Elligsen M, Daneman N. Predictive utility of prior positive urine cultures. Clin Infect Dis. 2014; 59(9):1265-1271. doi:10.1093/cid/ciu588
23. Dickstein Y, Geffen Y, Andreassen S, Leibovici L, Paul M. Predicting Antibiotic Resistance in Urinary Tract Infection Patients with Prior Urine Cultures. Antimicrob Agents Chemother. 2016; 60(8):4717-4721. doi:10.1128/AAC.00202-16.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

We claim:

1. A system for selecting an antimicrobial for a patient presenting with a urinary tract infection (UTI), the system comprising:
at least one hardware processor that is programmed to:
receive information indicative of a place of residence of the patient presenting with symptoms of the UTI;
receive information indicating that the patient has a known history of previous uropathogen resistance to a previously administered antimicrobial;
identify an algorithm to use to select an antimicrobial based on:
(i) the information indicative of the place of residence of the patient presenting with symptoms of the UTI;
(ii) the information indicating that the patient has a known history of previous uropathogen resistance to the previously administered antimicrobial; or
(iii) both of (i) and (ii)
select, using the algorithm, an antimicrobial that is predicted to be effective for treating the patient's UTI based on (i), (ii), or (iii);
generate a report based on the selection of the antimicrobial that is predicted to be effective for treating the patient's UTI using the algorithm; and
cause the report to be presented.

2. The system of claim 1, wherein the algorithm is configured to select an antimicrobial based on antimicrobial resistance associated with a geographic area with which the place of residence of the patient is associated.

3. The system of claim 2, wherein the information indicative of the place of residence of the patient comprises a ZIP code.

4. The system of claim 1, wherein the report comprises a map demarcated by ZIP code, the map depicting at least geographic areas associated with a plurality of ZIP codes including the ZIP code corresponding to the place of residence of the patient and one or more neighboring ZIP codes, and wherein each of the plurality of ZIP codes is coded to indicate a degree of resistance to the antimicrobial that is predicted to be effective for treating the second patient's UTI observed in residents of the geographic area corresponding to the respective ZIP code.

5. The system of claim 4, where the at least one hardware processor is further programmed to:
receive information indicative of associations between geographic areas, including at least the geographic area associated with the plurality of ZIP codes, and resistance to a plurality of particular antimicrobials including at least the antimicrobial that is predicted to be effective for treating the second patient's UTI; and
render the map based on the information indicative of associations between geographic areas and resistance to the plurality of particular antimicrobials.

6. The system of claim 5, where the at least one hardware processor is further programmed to:
receive information indicating that the patient's UTI was resistant to the antimicrobial that is predicted to be effective for treating the patient's UTI; and
update the information indicative of associations between geographic areas and resistance to a plurality of particular antimicrobials based on the information indicating that the patient's UTI was resistant to the antimicrobial that is predicted to be effective for treating the patient's UTI.

7. The system of claim 1, where the at least one hardware processor is further programmed to:
receive identifying information associated with the patient; and
retrieve, from an electronic medical record system using the identifying information associated with the patient, the information indicative of the place of residence of the patient and the information indicating that the patient has a known history of previous uropathogen resistance to the first antimicrobial.

8. The system of claim 7, wherein the system comprises the electronic medical record system.

9. The system of claim 1, wherein the report comprises a map demarcated by ZIP code, the map depicting at least geographic areas associated with a plurality of ZIP codes including a ZIP code corresponding to the place of residence of the patient and one or more neighboring ZIP codes, and wherein each of the plurality of ZIP codes is coded to indicate a degree of resistance to the antimicrobial that is predicted to be effective for treating the patient's UTI observed in residents of the geographic area corresponding to the respective ZIP code.

10. The system of claim 1, wherein the at least one hardware processor is programmed to:
receive information indicative of a place of residence of a first patient presenting with symptoms of a UTI;
receive information indicating that the first patient has a known history of previous uropathogen resistance to a first antimicrobial;
identify a first algorithm to use to select an antimicrobial based on the information indicating that the first patient has a known history of previous uropathogen resistance to the first antimicrobial;
select, using the first algorithm, an antimicrobial that is predicted to be effective for treating the first patient's UTI based at least in part on the information indicating that the first patient has a known history of previous uropathogen resistance to the first antimicrobial;
generate a first report based on the selection of the antimicrobial that is predicted to be effective for treating the first patient's UTI using the first algorithm, the first report comprising information indicating that the antimicrobial that is predicted to be effective based on a known history of previous uropathogen resistance;
cause the first report to be presented;
receive information indicative of a place of residence of a second patient presenting with symptoms of a UTI;
identify a second algorithm to use to select an antimicrobial based on the place of residence of the second patient;
select, using the second algorithm, an antimicrobial that is predicted to be effective for treating the second patient's UTI based at least in part on the information indicative of the place of residence of the second patient;
generate a second report based on the selection of the antimicrobial that is predicted to be effective for treating the second patient's UTI using the second algorithm, the second report comprising information indicating that the antimicrobial is predicted to be effective based on the second patient's place of residence;
cause the second report to be presented.

11. The system of claim 10, wherein the at least one hardware processor is further programmed to:
receive information indicative of a place of residence of a third patient presenting with symptoms of a UTI;
receive information indicating that the third patient previously received a prescription for a second antimicrobial;
identify the first algorithm to use to select an antimicrobial based on the information indicating that the third patient previously received a prescription for the second antimicrobial;
select, using the first algorithm, an antimicrobial that is predicted to be effective for treating the second patient's UTI based at least in part on the information indicating that the third patient previously received a prescription for the second antimicrobial;
generate a third report based on the selection of the antimicrobial that is predicted to be effective for treating the third patient's UTI using the first algorithm, the first report comprising information indicating that the antimicrobial that is predicted to be effective based on the previous prescription for the second antimicrobial; and
cause the third report to be presented.

12. The system of claim 10, wherein the second algorithm is configured to select an antimicrobial based on antimicrobial resistance associated with a geographic area with which the place of residence of the second patient is associated.

13. A method for selecting an antimicrobial for a patient presenting with a urinary tract infection (UTI), the method comprising:
(a) receiving information indicative of a place of residence of the patient presenting with symptoms of the UTI;
(b) receiving information indicating that the patient has a known history of previous uropathogen resistance to a previously administered antimicrobial;
(c) identifying an algorithm to use to select an antimicrobial based on:
(i) the information indicative of the place of residence of the patient presenting with symptoms of the UTI;
(ii) the information indicating that the patient has a known history of previous uropathogen resistance to the previously administered antimicrobial; or
(iii) both of (i) and (ii)
(d) selecting, using the algorithm, an antimicrobial that is predicted to be effective for treating the patient's UTI based on (i), (ii), or (iii);
(e) generating a report based on the selection of the antimicrobial that is predicted to be effective for treating the patient's UTI using the algorithm; and
(f) causing the report to be presented.

14. The method of claim 13, wherein the information indicative of the place of residence of the patient comprises a ZIP code.

15. The method of claim 13, wherein the report comprises a map demarcated by ZIP code, the map depicting at least geographic areas associated with a plurality of ZIP codes including the ZIP code corresponding to the place of residence of the patient and one or more neighboring ZIP codes, and wherein each of the plurality of ZIP codes is coded to indicate a degree of resistance to the antimicrobial that is predicted to be effective for treating the patient's UTI observed in residents of the geographic area corresponding to the respective ZIP code.

16. The method of claim 15, further comprising:
receiving information indicative of associations between geographic areas, including at least the geographic area associated with the plurality of ZIP codes, and resistance to a plurality of particular antimicrobials including at least the antimicrobial that is predicted to be effective for treating the second patient's UTI; and
rendering the map based on the information indicative of associations between geographic areas and resistance to the plurality of particular antimicrobials.

17. The method of claim 16, further comprising:
receiving information indicating that the patient's UTI was resistant to the antimicrobial that is predicted to be effective for treating the patient's UTI; and
updating the information indicative of associations between geographic areas and resistance to a plurality of particular antimicrobials based on the information indicating that the patient's UTI was resistant to the antimicrobial that is predicted to be effective for treating the patient's UTI.

18. The method of claim 13, further comprising:
receiving identifying information associated with the patient; and
retrieving, from an electronic medical record system using the identifying information associated with the patient, the information indicative of the place of residence of the patient and the information indicating that the patient has a known history of previous uropathogen resistance to the previously administered antimicrobial.

19. The method of claim 13, wherein the report comprises a map demarcated by ZIP code, the map depicting at least geographic areas associated with a plurality of ZIP codes including a ZIP code corresponding to the place of residence of the patient and one or more neighboring ZIP codes, and wherein each of the plurality of ZIP codes is coded to indicate a degree of resistance to the antimicrobial that is predicted to be effective for treating the patient's UTI observed in residents of the geographic area corresponding to the respective ZIP code.

20. The method of claim 13 comprising:
receiving information indicative of a place of residence of a first patient presenting with symptoms of a UTI;
receiving information indicating that the first patient has a known history of previous uropathogen resistance to a first antimicrobial;
identifying a first algorithm to use to select an antimicrobial based on the information indicating that the first patient has a known history of previous uropathogen resistance to the first antimicrobial;
selecting, using the first algorithm, an antimicrobial that is predicted to be effective for treating the first patient's UTI based at least in part on the information indicating that the first patient has a known history of previous uropathogen resistance to the first antimicrobial;
generating a first report based on the selection of the antimicrobial that is predicted to be effective for treating the first patient's UTI using the first algorithm, the first report comprising information indicating that the antimicrobial that is predicted to be effective based on a known history of previous uropathogen resistance;
causing the first report to be presented;
receiving information indicative of a place of residence of a second patient presenting with symptoms of a UTI;
identifying a second algorithm to use to select an antimicrobial based on the place of residence of the second patient;
selecting, using the second algorithm, an antimicrobial that is predicted to be effective for treating the second patient's UTI based at least in part on the information indicative of the place of residence of the second patient;
generating a second report based on the selection of the antimicrobial that is predicted to be effective for treating the second patient's UTI using the second algorithm, the second report comprising information indicating that the antimicrobial is predicted to be effective based on the second patient's place of residence;
causing the second report to be presented.

21.-24. (canceled)
